SEQUENCE LISTING (97 sequences)

SEQ ID NO: 1 - RNA, 56 nt, Alicyclobacillus acidoterrestris
  misc_feature (37)..(46): n is a, c, g, or u
  misc_feature (47)..(56): N is A, C, G, or U
gucggaucacugagcgagcgaucugagaaguggcacnnnnnnnnnnnnnnnnnnnn

SEQ ID NO: 2 - RNA, 42 nt, Alicyclobacillus acidoterrestris
  misc_feature (23)..(42): N is A, C, G, or U
cgagcgaucugagaaguggcacnnnnnnnnnnnnnnnnnnnn

SEQ ID NO: 3 - RNA, 34 nt, Alicyclobacillus acidoterrestris
  misc_feature (14)..(34): N is A, C, G, or U
cugagaaguggcacnnnnnnnnnnnnnnnnnnnn

SEQ ID NO: 4 - RNA, 79 nt, Alicyclobacillus acidoterrestris
gucuagaggacagaauuuuucaacgggugugccaauggccacuuuccagguggcaaagcccguugagcuucucaaaaag

SEQ ID NO: 5 - RNA, 78 nt, Alicyclobacillus acidoterrestris
gucuagaggacagaauuuuucaacgggugugccaauggccacuuuccagguggcaaagcccguugagcuucucaaaaa

SEQ ID NO: 6 - RNA, 67 nt, Alicyclobacillus acidoterrestris
gucuagaggacagaauuuuucaacgggugugccaauggccacuuuccagguggcaaagcccguugag

SEQ ID NO: 7 - RNA, 111 nt, Alicyclobacillus acidoterrestris
  misc_feature (92)..(111): N is A, C, G, or U
gucuagaggacagaauuuuucaacgggugugccaauggccacuuuccagguggcaaagcccguugagcuucucaaaucugagaaguggcacnnnnnnnnnnnnnnnnnnnn

SEQ ID NO: 8 - RNA, 104 nt, Artificial Sequence
  Synthetic
  misc_feature (85)..(104): N is A, C, G, or U
gucgucuauaggacggcgaggacaacgggagugcagugcucuuuccaagagcaaacaccccguuggcuucaagagaaguggcacnnnnnnnnnnnnnnnnnnnn

SEQ ID NO: 9 - RNA, 91 nt, Bacillus thermoamylovorans
cgagguucugucuuuuggucaggacaaccgucuagcuauaagugcugcaggggugugagaaacuccuauugcuggacgaugucucuuuuau

SEQ ID NO: 10 - RNA, 56 nt, Bacillus thermoamylovorans
  misc_feature (37)..(56): N is A, C, G, or U
guccaagaaaaaagaaaugauacgaggcauuagcacnnnnnnnnnnnnnnnnnnnn

SEQ ID NO: 11 - RNA, 42 nt, Bacillus thermoamylovorans
  misc_feature (23)..(42): N is A, C, G, or U
aaaugauacgaggcauuagcacnnnnnnnnnnnnnnnnnnnn

SEQ ID NO: 12 - RNA, 34 nt, Bacillus thermoamylovorans
  misc_feature (15)..(34): N is A, C, G, or U
cgaggcauuagcacnnnnnnnnnnnnnnnnnnnn

SEQ ID NO: 13 - RNA, 44 nt, Unknown
  Deltaproteobacteria
  misc_feature (24)..(44): N is A, C, G, or U
ccgauaaguaaaacgcaucaaagnnnnnnnnnnnnnnnnnnnnn

SEQ ID NO: 14 - RNA, 75 nt, Unknown
  Deltaproteobacteria
ggcgcguuuauuccauuacuuuggagccagucccagcgacuaugucguauggacgaagcgcuuauuuaucggaga

SEQ ID NO: 15 - RNA, 122 nt, Artificial Sequence
  Synthetic
  misc_feature (102)..(122): N is A, C, G, or U
ggcgcguuuauuccauuacuuuggagccagucccagcgacuaugucguauggacgaagcgcuuauuuaucggagagaaaccgauaaguaaaacgcaucaaannnnnnnnnnnnnnnnnnnnn

SEQ ID NO: 16 - RNA, 128 nt, Artificial Sequence
  Synthetic
  misc_feature (109)..(128): N is A, C, G, or U
acaucuggcgcguuuauuccauuacuuuggagccagucccagcgacuaugucguauggacgaagcgcuuauuuaucggagagaaaccgauaaguaaaacgcaucaaagnnnnnnnnnnnnnnnnnnnn

SEQ ID NO: 17 - RNA, 225 nt, Artificial Sequence
  Synthetic
  misc_feature (206)..(225): N is A, C, G, or U
gggcuucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuuuuccucuccaauucugcacaagaaaguugcagaacccgaauagacgaaugaaggaaugcaacnnnnnnnnnnnnnnnnnnnn

SEQ ID NO: 18 - RNA, 116 nt, Artificial Sequence
  Synthetic
cuucaccaaaagcugucccuuaggggauuagaacuugagugaaggugggcugcuugcaucagccuaaugucgagaagugcuuucuucggaaaguaacccucgaaacaaauucauuu

SEQ ID NO: 19 - RNA, 37 nt, Artificial Sequence
  Synthetic
  misc_feature (18)..(37): N is A, C, G, or U
gaaugaaggaaugcaacnnnnnnnnnnnnnnnnnnnn

SEQ ID NO: 20 - RNA, 57 nt, Artificial Sequence
  Synthetic
  misc_feature (38)..(57): N is A, C, G, or U
guugcagaacccgaauagacgaaugaaggaaugcaacnnnnnnnnnnnnnnnnnnnn

SEQ ID NO: 21 - RNA, 157 nt, Artificial Sequence
  Synthetic
  misc_feature (138)..(157): n is a, c, g, or u
cuucaccaaaagcugucccuuaggggauuagaacuugagugaaggugggcugcuugcaucagccuaaugucgagaagugcuuucuucggaaaguaacccucgaaacaaauucauuugaaagaaugaaggaaugcaacnnnnnnnnnnnnnnnnnnnn

SEQ ID NO: 22 - protein, 84 aa, Unknown
  Bacillus phage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 23 - RNA, 66 nt, Artificial Sequence
  Telomerase Ku biding motif
uucuugucguacuuauagaucgcuacguuauuucaauuuugaaaaucugaguccugggagugcgga

SEQ ID NO: 24 - protein, 608 aa, Artificial Sequence
  Ku heterodimer
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQ

SEQ ID NO: 25 - protein, 485 aa, Artificial Sequence
  Ku heterodimer
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 26 - RNA, 10 nt, Artificial Sequence
  Telomerase Sm7 binding motif
aauuuuugga

SEQ ID NO: 27 - protein, 83 aa, Artificial Sequence
  Sm consensus site
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 28 - RNA, 23 nt, Artificial Sequence
  MS2 phage operator stem loop
gcacaugaggaucacccaugugc

SEQ ID NO: 29 - protein, 117 aa, Artificial Sequence
  MS2 coat protein
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 30 - RNA, 26 nt, Artificial Sequence
  PP7 hage operator stem loop
auaaggaguuuauauggaaacccuua

SEQ ID NO: 31 - protein, 128 aa, Artificial Sequence
  PP7 coat protein PCP
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 32 - RNA, 19 nt, Artificial Sequence
  SfMu Com Stem Loop
cugaaugccugcgagcauc

SEQ ID NO: 33 - protein, 62 aa, Artificial Sequence
  SfMU Com binding protein
MKSIRCKNCNKLLFKADSFDHIEIRCPRCKRHIIMLNACEHPTEKHCGKREKITHSDETVRY

SEQ ID NO: 34 - RNA, 15 nt, Artificial Sequence
  Box B Aptamer
gcccugaagaagggc

SEQ ID NO: 35 - protein, 22 aa, Artificial Sequence
  Lambda N22plus protein
MNARTRRRERRAEKQAQWKAAN

SEQ ID NO: 36 - RNA, 16 nt, Artificial Sequence
  Csy4 binding motif
cugccguauaggcagc

SEQ ID NO: 37 - protein, 187 aa, Artificial Sequence
  Csy4[H29A]
MDHYLDIRLRPDPEFPPAQLMSVLFGKLAQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 38 - DNA, 147 bp, Artificial Sequence
  Synthetic
  misc_feature (127)..(147): N is A, C, G, T, or U
gcgcacatgaggatcacccatgtgcggcgcguuuauuccauuacuuuggagccagucccagcgacuaugucguauggacgaagcgcuuauuuaucggagagaaaccgauaaguaaaacgcaucaaannnnnnnnnnnnnnnnnnnnn

SEQ ID NO: 39 - DNA, 146 bp, Artificial Sequence
  Synthetic
  misc_feature (126)..(146): N is A, C, G, T, or U
ggcgcguuuauuccauuacuuuggagccagucccagcgacugcgcacatgaggatcacccatgtgcugucguauggacgaagcgcuuauuuaucggagagaaaccgauaaguaaaacgcaucaaannnnnnnnnnnnnnnnnnnnn

SEQ ID NO: 40 - DNA, 146 bp, Artificial Sequence
  Synthetic
  misc_feature (126)..(146): N is A, C, G, or U
ggcgcguuuauuccauuacuuuggagccagucccagcgacuaugucguauggacgaagcgcuuauuuaucggagagcgcacatgaggatcacccatgtgcaaaccgauaaguaaaacgcaucaaannnnnnnnnnnnnnnnnnnnn

SEQ ID NO: 41 - DNA, 147 bp, Artificial Sequence
  Synthetic
  misc_feature (102)..(122): N is A, C, G, T, or U
ggcgcguuuauuccauuacuuuggagccagucccagcgacuaugucguauggacgaagcgcuuauuuaucggagagaaaccgauaaguaaaacgcaucaaannnnnnnnnnnnnnnnnnnnngcgcacatgaggatcacccatgtgc

SEQ ID NO: 42 - DNA, 171 bp, Artificial Sequence
  Synthetic
  misc_feature (151)..(171): N is A, C., G, T,  or U
gcgcacatgaggatcacccatgtgcggcgcguuuauuccauuacuuuggagccagucccagcgacuaugucguauggacgaagcgcuuauuuaucggagagcgcacatgaggatcacccatgtgcaaaccgauaaguaaaacgcaucaaannnnnnnnnnnnnnnnnnnnn

SEQ ID NO: 43 - DNA, 20 bp, Homo sapiens
agaaccggaggacaaagtac

SEQ ID NO: 44 - DNA, 20 bp, Homo sapiens
tggcaatgcgccaccggttg

SEQ ID NO: 45 - DNA, 20 bp, Homo sapiens
tcttctgctcggactcaggc

SEQ ID NO: 46 - DNA, 20 bp, Homo sapiens
tctgctcggactcaggccct

SEQ ID NO: 47 - DNA, 20 bp, Homo sapiens
ccagcttctgccgtttgtac

SEQ ID NO: 48 - DNA, 20 bp, Homo sapiens
aaaaacagtggataattttg

SEQ ID NO: 49 - DNA, 20 bp, Homo sapiens
gaagagaccaaagatcaccc

SEQ ID NO: 50 - DNA, 20 bp, Homo sapiens
cctccgcctgtggatgctgc

SEQ ID NO: 51 - DNA, 20 bp, Homo sapiens
tcctgctgctgccgggacct

SEQ ID NO: 52 - DNA, 20 bp, Homo sapiens
gcggccgatgagaagaagaa

SEQ ID NO: 53 - DNA, 69 bp, Artificial Sequence
  Synthetic
  misc_feature (49)..(69): N is A, C, G, T, or U
gcgcacatgaggatcacccatgtgcccgauaaguaaaacgcaucaaagnnnnnnnnnnnnnnnnnnnnn

SEQ ID NO: 54 - DNA, 69 bp, Artificial Sequence
  Synthetic
  misc_feature (24)..(44): N is A, C, G, T, or U
ccgauaaguaaaacgcaucaaagnnnnnnnnnnnnnnnnnnnnngcgcacatgaggatcacccatgtgc

SEQ ID NO: 55 - DNA, 100 bp, Artificial Sequence
  Synthetic
gcgcacatgaggatcacccatgtgcggcgcguuuauuccauuacuuuggagccagucccagcgacuaugucguauggacgaagcgcuuauuuaucggaga

SEQ ID NO: 56 - DNA, 99 bp, Artificial Sequence
  Synthetic
ggcgcguuuauuccauuacuuuggagccagucccagcgacugcgcacatgaggatcacccatgtgcugucguauggacgaagcgcuuauuuaucggaga

SEQ ID NO: 57 - DNA, 100 bp, Artificial Sequence
  Synthetic
ggcgcguuuauuccauuacuuuggagccagucccagcgacuaugucguauggacgaagcgcuuauuuaucggagagcgcacatgaggatcacccatgtgc

SEQ ID NO: 58 - DNA, 118 bp, Artificial Sequence
  Synthetic
  misc_feature (92)..(111): N is A, C, G, U, or T
gtctagaggacagaatttttcaacgggtgtgccaatggccactttccaggtggcaaagcccgttgagcttctcaaatctgagaagtggcacnnnnnnnnnnnnnnnnnnnnttttttt

SEQ ID NO: 59 - DNA, 142 bp, Artificial Sequence
  Synthetic
  misc_feature (116)..(135): N is A, C, G, or T
ggcacatgaggatcacccatgtgcgtctagaggacagaatttttcaacgggtgtgccaatggccactttccaggtggcaaagcccgttgagcttctcaaatctgagaagtggcacnnnnnnnnnnnnnnnnnnnnttttttt

SEQ ID NO: 60 - DNA, 142 bp, Artificial Sequence
  Synthetic
  misc_feature (116)..(135): N is A, C, G, or T
ggcacatgaggatcacccatgtgcgtctagaggacagaattgttcaacgggtgtgccaatggccactttccaggtggcaaagcccgttgagcttctcaaatctgagaagtggcacnnnnnnnnnnnnnnnnnnnnttttttt

SEQ ID NO: 61 - DNA, 142 bp, Artificial Sequence
  Synthetic
  misc_feature (116)..(135): N is A, C, G, or T
ggtctagaggacagaatttttcaacgggtgtgccaatggccactttccaggtggcaaagcccgttgagcttctcaaatctgagaagtggcacgcacatgaggatcacccatgtgcnnnnnnnnnnnnnnnnnnnnttttttt

SEQ ID NO: 62 - DNA, 142 bp, Artificial Sequence
  Synthetic
  misc_feature (116)..(135): N is A, C, G, or T
ggtctagaggacagaattgttcaacgggtgtgccaatggccactttccaggtggcaaagcccgttgagcttctcaaatctgagaagtggcacgcacatgaggatcacccatgtgcnnnnnnnnnnnnnnnnnnnnttttttt

SEQ ID NO: 63 - DNA, 131 bp, Artificial Sequence
  Synthetic
  misc_feature (105)..(124): N is A, C, G, or T
ggtctagaggacagaatttttcaacgggtgtgccaatggacatgaggatcacccatgtccaggtggcaaagcccgttgagcttctcaaatctgagaagtggcacnnnnnnnnnnnnnnnnnnnnttttttt

SEQ ID NO: 64 - DNA, 131 bp, Artificial Sequence
  Synthetic
  misc_feature (105)..(124): N is A, C, G, or T
ggtctagaggacagaattgttcaacgggtgtgccaatggacatgaggatcacccatgtccaggtggcaaagcccgttgagcttctcaaatctgagaagtggcacnnnnnnnnnnnnnnnnnnnnttttttt

SEQ ID NO: 65 - DNA, 142 bp, Artificial Sequence
  Synthetic
  misc_feature (116)..(135): N is A, C, G, or T
ggtctagaggacagaattgcacatgaggatcacccatgtgctttcaacgggtgtgccaatggccactttccaggtggcaaagcccgttgagcttctcaaatctgagaagtggcacnnnnnnnnnnnnnnnnnnnnttttttt

SEQ ID NO: 66 - DNA, 192 bp, Artificial Sequence
  Synthetic
  misc_feature (163)..(185): N is A, C, G, or T
atgggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnnnttttttt

SEQ ID NO: 67 - DNA, 215 bp, Artificial Sequence
  Synthetic
  misc_feature (186)..(208): N is A, C, G, or T
gcacatgaggatcacccatgtgcatgggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnnnttttttt

SEQ ID NO: 68 - DNA, 188 bp, Artificial Sequence
  Synthetic
  misc_feature (159)..(181): N is A, C, G, or T
gcacatgaggatcacccatgtgccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnnnttttttt

SEQ ID NO: 69 - DNA, 158 bp, Artificial Sequence
  Synthetic
  misc_feature (129)..(151): N is A, C, G, or T
accgcttcacgcacatgaggatcacccatgtgcgtgaaggtgggctgcttgcatcagcctaatgtcgagaagtgctttcttcggaaagtaaccctcgaaacaaattcatttgaatgaaggaatgcaacnnnnnnnnnnnnnnnnnnnnnnnttttttt

SEQ ID NO: 70 - DNA, 158 bp, Artificial Sequence; identical to SEQ ID NO: 69
  Synthetic
  misc_feature (129)..(151): N is A, C, G, or T

SEQ ID NO: 71 - DNA, 199 bp, Artificial Sequence
  Plasmid containing sequences from Cas12b, SV40 NLS
  misc_feature (170)..(192): N is A, C, G, or T
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaagcacatgaggatcacccatgtgcggaatgcaacnnnnnnnnnnnnnnnnnnnnnnnttttttt

SEQ ID NO: 72 - DNA, 4035 bp, Artificial Sequence
  Plasmid containing sequences from humanized Cas12b and SV40 NLS
atggccccaaagaagaagcggaaagtcgccgtgaaaagcattaaagtgaaactgaggctggacgatatgcccgaaatcagagccggcctctggaagctgcacaaggaggtcaacgccggcgttcgttattacacagagtggctgtctttactcagacaagaaaatttatatagaaggagccccaatggcgatggcgagcaagagtgcgacaaaaccgccgaagaatgcaaggccgaactgctcgaaagactgagagccagacaagttgagaacggacacagaggacccgccggctccgatgatgaactgctgcagctcgctaggcaactgtacgagctgctcgtcccccaagctatcggagctaaaggagacgctcagcagatcgctaggaagtttctgagccccctcgccgataaagacgctgtgggcggtttaggaatcgccaaggctggaaataaacctcgttgggtgaggatgagggaagctggcgagcccggctgggaggaggaaaaggagaaagccgaaactcgtaaatccgccgacagaacagctgacgtgctgagggccctcgccgacttcggtttaaaacccctcatgagagtctataccgactccgagatgtccagcgtcgagtggaaacccttacgtaagggccaagctgtgagaacatgggatcgtgatatgttccagcaagctatcgaaaggatgatgagctgggagtcttggaatcagagagtgggccaagaatacgccaaactcgtggaacagaagaatcgtttcgagcagaaaaactttgtcggacaagaacatttagtgcatctcgtgaaccaactgcagcaagatatgaaggaggcctcccccggtttagagagcaaagagcaaaccgctcactatgtcaccggtcgtgccctcagaggctccgacaaagtgttcgaaaagtggggaaagctggcccccgacgctcctttcgatttatacgacgccgagatcaagaacgtgcagaggagaaatacaaggaggtttggctcccacgatttattcgctaaactcgccgagcccgaataccaagctttatggagagaggatgccagctttctgactcgttacgccgtgtacaattccattctgagaaaactgaaccacgccaaaatgtttgccacattcactttacccgatgccaccgcccaccctatctggacaagattcgacaagctcggcggaaacctccaccagtatacctttttatttaacgaattcggagagaggaggcatgccattcgttttcacaagttattaaaggtcgagaatggagtcgccagagaggtggacgacgtcaccgtgcctatcagcatgagcgaacagctggacaatttactgcctcgtgaccccaacgaacctatcgccctctacttcagagactacggagctgagcagcatttcaccggcgagttcggaggcgctaagatccagtgtagaagggatcaactggcccatatgcataggagaaggggcgccagagatgtctatctgaacgtgagcgttcgtgtccaaagccaaagcgaggccagaggagaaaggagacccccctacgccgccgtctttagactggtcggcgacaatcatcgtgctttcgtgcactttgataagctgtccgactatttagccgaacaccccgacgatggaaagctgggcagcgagggattattaagcggcctcagagtcatgagcgtggctctgggcctcagaaccagcgcctccatctccgtctttagggtggccagaaaagacgagctgaagcccaacagcaagggaagggtgccctttttcttccctatcaagggcaatgacaatttagtggccgtgcacgaaaggtcccagttattaaagctgcccggcgagaccgaaagcaaagatttaagggctatcagagaggagagacagagaactttaagacagctgagaacccagctggcttatctgagattattagtcagatgtggcagcgaggacgtcggtcgtagagagaggagctgggccaagctgattgaacaacccgttgatgccgctaatcacatgacccccgattggagggaagctttcgagaacgagctgcagaaactgaagtctttacacggcatttgcagcgacaaggagtggatggacgccgtgtacgagtccgtgagaagggtgtggaggcacatgggaaagcaagttagggattggaggaaagatgtgaggtccggcgaaagacccaagatcagaggctacgccaaggacgtggtcggaggaaactccatcgagcagatcgagtacctcgagagacaatacaagtttttaaagtcttggtccttcttcggcaaggtcagcggccaagtcattcgtgctgaaaagggatctcgtttcgccatcacactgagagagcacattgaccacgccaaagaggatcgtctgaaaaaactcgccgatcgtatcattatggaggccctcggctatgtctatgctctggacgagagaggcaagggaaaatgggtcgccaagtatcccccttgtcaactgattttattagcggagctgtccgagtaccaatttaacaacgataggcctccctccgagaataaccagctcatgcagtggagccatcgtggcgtgtttcaagaactgatcaatcaagctcaagttcacgatttactcgtgggcaccatgtacgccgcttttagctccagattcgacgctaggaccggcgcccccggtattaggtgtagaagagtgcccgctcgttgcacccaagaacataaccccgaaccctttccttggtggctgaataagttcgtcgtcgagcacaccctcgacgcttgccctttacgtgccgacgacctcatccctactggtgaaggcgagatctttgtgagccctttcagcgctgaggaaggcgattttcaccagatccacgccgctctgaacgccgcccagaatctgcaacagaggctctggagcgacttcgatatttcccagattcgtctgaggtgcgattggggagaggtggatggagaactcgtcctcatccccagactgactggtaagaggaccgctgacagctattccaataaggtcttctataccaatactggtgtcacctactacgagagagagaggggcaagaagagaaggaaagtcttcgcccaagagaagctgtccgaggaggaggccgaattattagtcgaggctgatgaggctcgtgaaaagtccgtggttttaatgagggacccctccggcatcatcaacagaggcaattggactcgtcagaaggaattctggagcatggtgaatcagaggatcgagggctatctggtgaagcagattagatctcgtgtgcctctgcaagatagcgcttgtgagaataccggagatattagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctaatgcttcccgaagaagtcgaagaagtgatcggaaacaagcctgagagcgatatcctggtccatactgcgtatgatgaaagtaccgacgaaaacgtaatgctactcacatccgacgccccagagtataagccctgggctctagttatacaagactccaacggagagaacaaaatcaaaatgctgtctggcggctcaaaaagaaccgccgacggcagcgaattcgagcccaagaagaagaggaaagtctaa

SEQ ID NO: 73 - protein, 1344 aa, Artificial Sequence
  Protein containing sequences from Cas12b and SV40 NLS
MAPKKKRKVAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVALGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLAELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHAALNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDISGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 74 - DNA, 2235 bp, Artificial Sequence
  Plasmid containing sequences from humanized CasMINI and SV40 NLS
atggcccccaagaaaaaacgcaaggtggccaaaaacaccattaccaaaacactgaaactgcgtattgtgcgtccgtataatagcgcagaagtggaaaaaattgttgccgacgaaaaaaacaaccgcgaaaaaatcgcactggaaaagaacaaagacaaagtgaaagaagcctgcagcaaacatctgaaagttgcagcatattgtaccacacaggttgaacgtaatgcatgcctgttttgtaaagcacgtaaactggatgacaaattctaccaaaaactgcgtggtcagtttccggatgcagttttttggcaagaaatcagcgaaatttttcgccagctgcagaaacaggcagcagaaatctataatcagagcctgatcgaactgtactacgagatttttatcaaaggcaaaggtattgcaaatgccagcagcgttgaacattatctgagtagagtttgttatagacgtgcagcagaactgtttaaaaacgcagcaattgcaagcggtctgcgtagcaaaatcaaaagcaattttcgtctgaaagaactgaaaaacatgaaaagtggtctgccgaccaccaaaagcgataattttccgattccgctggttaaacagaaaggtggtcagtataccggttttgaaattagcaatcataatagcgacttcatcatcaagattccgtttggtcgttggcaggtcaaaaaagagattgataaatatcgtccgtgggagaaatttgactttgaacaggttcagaaaagcccgaaaccgattagcctgctgctgagcacccagcgtcgtaaacgtaataaaggttggagcaaagatgaaggcaccgaagccgaaatcaaaaaagttatgaatggcgattatcagaccagctacattgaagttaaacgtggcagcaaaatctgtgaaaaaagcgcatggatgctgaatctgagcattgatgttccgaaaattgataaaggtgtggatccgagcattattggtggtattgcagttggtgttagatcaccgctggtttgcgcaattaacaatgcatttagccgttatagcatcagcgataacgacctgtttcacttcaacaagaaaatgtttgcacgtcgtcgtatcctgctgaaaaaaaaccgtcataaacgtgcaggtcatggtgcaaaaaacaaactgaaaccgatcaccattctgaccgaaaaaagtgaacgttttcgcaaaaagctgattgaacgttgggcatgtgaaatcgcggatttcttcattaaaaacaaagttggcaccgtgcagatggaaaatctggaaagcatgaaacgtaaagaggacagctattttaacattcgcctgcgtggcttttggccgtatgcagaaatgcagaacaaaatcgaattcaaactgaagcagtatggcatcgaaattcgtaaagttgcaccgaataataccagcaaaacctgtagcaaatgtggccatctgaacaactatttcaacttcgagtaccgcaagaaaaacaaattcccgcactttaaatgcgaaaaatgcaacttcaaagaaaacgccgcgtataatgcagccctgaatatttcaaacccgaaactgaaaagcaccaaagagagaccgagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaa

SEQ ID NO: 75 - protein, 744 aa, Artificial Sequence
  Protein containing sequences from CasMINI and SV40 NLS
MAPKKKRKVAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 76 - DNA, 1050 bp, Artificial Sequence
  Plasmid containing sequences from human Apobec3A and SV40 NLS
atggcccccaagaagaagcggaaagtggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccaaatctacagggtcacttggttcatctcctggagcccctgcttctccgcgggctgtgccggggaagtgcgtgcgttccttcaggagaacacacacgtgagactgcgtatcttcgctgcccgcatctatgatgacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaacgagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctactga

SEQ ID NO: 77 - protein, 349 aa, Artificial Sequence
  Protein containing sequences from human Apobec3A and SV40 NLS
MAPKKKRKVEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSAGCAGEVRAFLQENTHVRLRIFAARIYDDDPLYKEALQMLRDAGAQVSIMTYDEFKHCWDTFVDHQGCPFQPWDGLDEHSQALSGRLRAILQNQGNELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 78 - DNA, 1008 bp, Artificial Sequence
  Plasmid containing sequences from Anolis Apobec1a and SV40 NLS
atggcccccaagaagaagcggaaagtggggtatcaggctgcaattctattatcaaatttgttcttcaggtggcaaatggaaccagaggcgtttcagaggaattttgatcccagagaatttcccgagtgtactttactgctgtatgaaatccactgggataacaacaccagtaggaactggtgtacaaacaaacctggcctccatgctgaagaaaattttttgcaaatttttaatgagaaaatagatatcaggcaggacacaccatgctctatcacttggtttctgtcttggagtccttgttatccatgcagccaggctataattaagttcttggaagcacaccctaacgtgagcctggagataaaagctgctcggctgtacatgcatcaaatcgactgtaacaaggaaggcctcaggaatttaggtagaaatagagtttctatcatgaatctacctgattatcgccactgttggacaacatttgtggttcctagaggggcaaatgaagattattggccacaggatttcttaccagccataacaaattattccagggaacttgactcaattcttcaggacgagctgaagacacccctgggcgacaccacacacacctctccaccttgcccagcaccagagctgctgggaggccctatggccagcaacttcacacagtttgtgctggtggataatggaggaaccggcgacgtgacagtggcaccatctaactttgccaatggcatcgccgagtggatcagctccaactctcggagccaggcctataaggtgacctgtagcgtgcggcagtctagcgcccagaatagaaagtatacaatcaaggtggaggtgcctaagggcgcctggagatcctacctgaacatggagctgaccatcccaatctttgccacaaattctgattgcgagctgatcgtgaaggccatgcagggcctgctgaaggacggcaaccctatcccaagcgccatcgccgccaatagcggaatctactga

SEQ ID NO: 79 - protein, 335 aa, Artificial Sequence
  Protein containing sequences from Anolis Apobec1a and SV40 NLS
MAPKKKRKVGYQAAILLSNLFFRWQMEPEAFQRNFDPREFPECTLLLYEIHWDNNTSRNWCTNKPGLHAEENFLQIFNEKIDIRQDTPCSITWFLSWSPCYPCSQAIIKFLEAHPNVSLEIKAARLYMHQIDCNKEGLRNLGRNRVSIMNLPDYRHCWTTFVVPRGANEDYWPQDFLPAITNYSRELDSILQDELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 80 - DNA, 20 bp, Homo sapiens
tgttccagtttcctttacag

SEQ ID NO: 81 - DNA, 20 bp, Homo sapiens
cagcccgctggccctgtaaa

SEQ ID NO: 82 - DNA, 20 bp, Homo sapiens
gccagagccggggtgtgcag

SEQ ID NO: 83 - DNA, 20 bp, Homo sapiens
ggaagtgtccagggatgctt

SEQ ID NO: 84 - DNA, 20 bp, Homo sapiens
tcgcgccctcccagccgggc

SEQ ID NO: 85 - DNA, 23 bp, Homo sapiens
ctcctggaccccctatttctgac

SEQ ID NO: 86 - DNA, 23 bp, Homo sapiens
gccagagccggggtgtgcagacg

SEQ ID NO: 87 - DNA, 20 bp, Artificial Sequence
  Synthetic
aggacgtctgcccaatatgt

SEQ ID NO: 88 - DNA, 20 bp, Artificial Sequence
  Synthetic
ccaagtgagaagccagtgga

SEQ ID NO: 89 - DNA, 20 bp, Artificial Sequence
  Synthetic
tcttccctcccagtcactga

SEQ ID NO: 90 - DNA, 20 bp, Artificial Sequence
  Synthetic
tcactctcgaagacgctgct

SEQ ID NO: 91 - DNA, 22 bp, Artificial Sequence
  Synthetic
cgatgaggagacactccaagag

SEQ ID NO: 92 - DNA, 22 bp, Artificial Sequence
  Synthetic
gcctttctgaaggtcatagtgc

SEQ ID NO: 93 - DNA, 20 bp, Homo sapiens
aggctggcccgccccgcagt

SEQ ID NO: 94 - DNA, 20 bp, Homo sapiens
ccgatattcctcaggtactc

SEQ ID NO: 95 - DNA, 20 bp, Homo sapiens
aggtttactcacgtcatcca

SEQ ID NO: 96 - DNA, 19 bp, Unknown
  Qbeta phage operator stem loop
atgctgtctaagacagcat

SEQ ID NO: 97 - protein, 133 aa, Unknown
  Qbeta coat protein
MAKLETVTLGNIGKDGKQTLVLNPRGVNPTNGVASLSQAGAVPALEKRVTVSVSQPSRNRKNYKVHVKIQNPTACTANGSCDPSVTRQAYADVTFSFTQYSTDEERAFVRTELAALLASPLLIDAIDQLNPAY

(19) United States
(12) Patent Application Publication
(10) Pub. No.: US 2024/0060088 A1
(43) Pub. Date: Feb. 22, 2024

(54) Guide RNA Designs and Complexes for Type V Cas Systems

(71) Applicant: Horizon Discovery Limited, Waterbeach, Cambridge (GB)

(72) Inventors: Hide Bueno Machado, Arvada, CO (US); Kevin Hemphill, Erie, CO (US); Robert Blassberg, Hertford, Hertfordshire (GB); Amanda Smith, Trumpington, Cambridge (GB); Michael Daniel Rushton, Hitchin, Hertfordshire (GB); Pablo Perez-Duran, Cambridge (GB)

(21) Appl. No.: 18/270,747

(22) PCT Filed: Jan. 5, 2022
(86) PCT No.: PCT/US2022/011294

§ 371 Date: Jul. 3, 2023

Related U.S. Application Data
(60) Provisional application No. 63/133,945, filed on Jan. 5, 2021.

(51) Int. Cl. C12N 15/90; C12N 9/22; C12N 9/78; C12N 15/11; C12N 15/85

(52) U.S. Cl. CPC C12N 15/90; C12N 9/22; C12N 9/78; C12N 15/11; C12N 15/85; C12Y 305/04; C12N 2310/20; C12N 2310/531; C12N 2800/107

(57) ABSTRACT

A novel gRNA-ligand binding complex is provided. This complex may be used to bring Type V Cas proteins and additional effectors to DNA for base editing. The design of the systems allows for the production of efficient modular components that provide flexibility when editing DNA.

### CROSS-REFERENCE TO RELATED APPLICATION

This application is a national stage application of international application serial number PCT/US2022/011294, filed Jan. 5, 2022, which claims the benefit of the filing date of U.S. Provisional Application Ser. No. 63/133,945, filed Jan. 5, 2021, the entire disclosures of which are incorporated by reference as if set forth fully herein.

### FIELD OF THE INVENTION

The present invention relates to the field of gene-editing.

### BACKGROUND OF THE INVENTION

Researchers are aggressively exploring the use of Clustered Regularly Interspaced Short Palindromic Repeat (CRISPR) systems in order to modify DNA. To date, the vast majority of the work in this field has been in Cas9 systems. In these systems, a tracrRNA (trans-activating CRISPR RNA) and a crRNA (CRISPR RNA) that are either in the form of two separate strands of nucleotides or regions of a single strand of nucleotides hybridize to recruit a Cas9 protein and then direct the Cas9 protein to a DNA location that is complementary to a sequence within the crRNA. The complementary sequence within the DNA thus becomes a target site, and the Cas9 protein may, based on its functional domain, cause editing at this target site.

Despite the now well recognized power of the Cas9 systems, those systems are not effective in all applications. Among the limitations of Cas9 systems is that the functional domains upon which the Cas9 systems can act are defined by the functional domain of the Cas9 protein that one uses.

Other Cas proteins are known. Among these other Cas proteins, the potential of which has not been fully explored, are those within the Type V family. The use of enzymes from the Type V family is particularly underexplored when one seeks to introduce multiple edits at or near a target site. Therefore, there is a need to develop improved gRNAs (guide RNAs), as well as complexes and systems that incorporate and use them.

### SUMMARY OF THE INVENTION

The present invention provides novel and non-obvious gRNA-ligand binding complexes, base editing complexes, and methods for base editing and genome modification. Through the use of various embodiments of the present invention, one may be able to efficiently and effectively cause base editing ex vivo, in vitro, and in vivo. Further, some embodiments of the present invention provide modular designs that allow for the same Type V Cas protein to be directed to different targeting sites and optionally associated with different effector proteins at the same or different sites.

According to a first embodiment, the present invention provides a gRNA-ligand binding complex, wherein the gRNA-ligand binding complex comprises: (a) a gRNA, wherein the gRNA contains 60 to 210 nucleotides or 80 to 180 nucleotides and the gRNA comprises (i) a crRNA sequence, wherein the crRNA sequence is 36 to 60 nucleotides long or 35 to 60 nucleotides long and the crRNA sequence comprises a Cas association region, wherein the Cas association region is 18 to 30 nucleotides long and a targeting region, wherein the targeting region is 18 to 30 nucleotides long, and (ii) a tracrRNA sequence, wherein the tracrRNA sequence is 45 to 120 nucleotides long and wherein the tracrRNA sequence comprises an anti-repeat region and a distal region, wherein the anti-repeat region is at least 80% complementary to the Cas association region over at least 18 consecutive nucleotides of the Cas association region and the Cas association region and the anti-repeat region are capable of hybridizing to form a hybridization region, wherein the hybridization region is capable of retaining association with an RNA binding domain of a Type V Cas protein; and (b) a ligand binding moiety, wherein the ligand binding moiety is either (i) directly bound to the gRNA, or (ii) bound to the gRNA through a linker.

According to a second embodiment, the present invention provides a base editing complex comprising: a gRNA-ligand binding complex of the present invention and a Type V Cas protein, wherein the Cas association region and the anti-repeat region of the gRNA-ligand binding complex are associated with the Type V Cas protein. Optionally, the ligand binding moiety is reversibly associated with a ligand that is attached to or a part of an effector molecule.

According to a third embodiment, the present invention provides a method for base editing. The method comprises exposing a base editing complex of the present invention to double stranded DNA (“dsDNA”) or to single stranded DNA (“ssDNA”). The base editing complex may be exposed to the dsDNA or ssDNA under conditions that permit base editing.

According to a fourth embodiment, the gRNA-ligand binding complex comprises or encodes SEQ ID NO: 28.

According to a fifth embodiment, the gRNA-ligand binding complex comprises or encodes any of SEQ ID NO: 59 to SEQ ID NO: 65.

According to a sixth embodiment, the gRNA-ligand binding complex comprises or encodes any of SEQ ID NO: 67 to SEQ ID NO: 71.

When an effector is attached to (or contains) a ligand, the system has a modular design. The presence of the ligand binding moiety within the gRNA-ligand binding complex allows that complex to associate with the corresponding ligand associated with (or contained within) the effector. Thus, the ligand binding moiety is associated with the gRNA in a manner and orientation that allows it to be capable of associating with a ligand. Similarly, the ligand is attached to or associated with the effector in a manner that renders it capable of reversibly associating with the ligand binding moiety.

When the ligand and the ligand binding moiety are associated with each other, the effector that is associated with the ligand will become part of any base editing complex that contains the gRNA-ligand binding complex. When the base editing complex also contains a Cas protein, that Cas protein and the effector can be retained in the same locality, e.g., at or near a target site of interest.

Thus, if one wishes to use a particular effector with the Cas protein, one only needs to associate that effector with the ligand that is capable of reversibly associating with the ligand binding moiety that is part of the base editing complex that contains that Cas protein. To change the effector from one system to the next, one need only change the effector-ligand. Consequently, one can use the same gRNA-ligand binding complex and its associated Cas protein with a plurality of different effectors. The plurality of different effectors may be used sequentially in the same system by associating and dissociating their ligands with the ligand binding moieties or simultaneously or sequentially in different systems.

### BRIEF DESCRIPTION OF THE FIGURES

FIGS. 1A-1D are representations of a Cas12b gRNA modified with one or two ligand binding moieties in which the tracrRNA and the crRNA are separate strands of oligonucleotides.

FIGS. 2A-2D are representations of a Cas12b gRNA modified with one or two ligand binding moieties in which the tracrRNA and the crRNA are part of the same strand of oligonucleotides.

FIGS. 3A-3D are representations of a Cas12e gRNA modified with a ligand binding moiety at different locations in which the tracrRNA and the crRNA are separate strands of oligonucleotides.

FIGS. 4A-4C are representations of a Cas12e gRNA modified with ligand binding moieties in which the tracrRNA and the crRNA are separate strands of oligonucleotides.

FIGS. 5A-5D are representations of a Cas12e gRNA modified with a ligand binding moiety at different locations in which the tracrRNA and the crRNA are part of the same strands of oligonucleotides.

FIGS. 6A-6C are representations of a Cas12f gRNA modified with a

ligand binding moiety at different locations in which the tracrRNA and the crRNA are separate strands of oligonucleotides.

FIGS. 7A-7C are representations of a Cas12f gRNA modified with one or two ligand binding moieties in which the tracrRNA and the crRNA are part of the same strand of oligonucleotides.

FIGS. 8A and 8B are bar graphs that show the percentage of C to T transitions that were introduced by a Cas12b base editor in an HEK-293T cell line with different effectors.

FIGS. 9A-9G are bar graphs that show the percentage of C to T transitions that were introduced by a Cas12b base editor in an HEK-293T cell line at multiple sites.

FIG. 10 is a bar graph that shows the percentage of C to T transitions that were introduced by a Cas12b base editor in a U2OS cell line.

FIGS. 11A-11D are bar graphs that show the percentage of C to T transitions that were introduced by a CasMINI base editor in an HEK-293T cell line.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

### Definitions

Unless otherwise stated or apparent from context, the following terms shall have the meanings set forth below:

The phrase “2′ modification” refers to a nucleotide unit having a sugar moiety that is modified at the 2′ position of the sugar moiety. An example of a 2′ modification is a 2′-O-alkyl modification that forms a 2′-O-alkyl modified nucleotide or a 2′ halogen modification that forms a 2′ halogen modified nucleotide.

The phrase “2′-O-alkyl modified nucleotide” refers to a nucleotide unit having a sugar moiety, for example a deoxyribosyl or ribosyl moiety that is modified at the 2′ position such that an oxygen atom is attached both to the carbon atom located at the 2′ position of the sugar and to an alkyl group. In various embodiments, the alkyl moiety consists of or consists essentially of carbon(s) and hydrogens. When the O moiety and the alkyl group to which it is attached are viewed as one group, they may be referred to as an O-alkyl group, e.g., —O-methyl, —O-ethyl, —O-propyl, —O-isopropyl, —O-butyl, —O-isobutyl, —O-ethyl-O-methyl (—OCH₂CH₂OCH₃), and —O-ethyl-OH (—OCH₂CH₂OH). A 2′-O-alkyl modified nucleotide may be substituted or unsubstituted.

The phrase “2′ halogen modified nucleotide” refers to a nucleotide unit having a sugar moiety, for example a deoxyribosyl moiety that is modified at the 2′ position such that the carbon at that position is directly attached to a halogen species, e.g., Fl, Cl, or Br.

A “ligand binding moiety” refers to a moiety such as an aptamer e.g., oligonucleotide or peptide or another compound that binds to a specific ligand and can reversibly or irreversibly be associated with that ligand.

The term “modified nucleotide” refers to a nucleotide having at least one modification in the chemical structure of the base, sugar and/or phosphate, including, but not limited to, 5-position pyrimidine modifications, 8-position purine modifications, modifications at cytosine exocyclic amines, and substitution of 5-bromo-uracil or 5-iodouracil; and 2′-modifications, including but not limited to, sugar-modified ribonucleotides in which the 2′-OH is replaced by a group such as an H, OR, R, halo, SH, SR, NH₂, NHR, NR₂, or CN.

Modified bases refer to nucleotide bases such as, for example, adenine, guanine, cytosine, thymine, uracil, xanthine, inosine, and queuosine that have been modified by the replacement or addition of one or more atoms or groups. Some examples of these types of modifications include, but are not limited to, alkylated, halogenated, thiolated, aminated, amidated, or acetylated bases, alone and in various combinations. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the position, 5-(2-amino)propyluridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O— and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4-thioribose, and other sugars, heterocycles, or carbocycles.

The phrase “codes for” and the term “encodes” mean that one sequence contains either a sequence that is identical to a referenced nucleotide sequence, a DNA or RNA equivalent of the referenced nucleotide sequence, or a DNA or RNA or a sequence that is a DNA or RNA complement of the referenced nucleotide sequence. Thus, when one refers to a sequence that codes for or encodes a recited DNA sequence, one refers to a sequence that unless otherwise specified is any one of the following: the same DNA sequence, a complement of the DNA sequence, the RNA equivalent of that sequence, or the RNA complement of that sequence or any of the aforementioned in which one or more ribonucleotides is substituted for its deoxyribonucleotide counterpart or one or more deoxyribonucleotides is substituted for its ribonucleotide counterpart.

The term “complementarity” refers to the ability of a nucleic acid to form one or more hydrogen bonds with another nucleic acid sequence by either traditional Watson-Crick base-pairing or other non-traditional types of base pairs. A percent complementarity indicates the percentage of residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all of the contiguous residues of a nucleic acid sequence will hydrogen bond with the same number of contiguous residues in a second nucleic acid sequence. “Substantially complementary” as used herein refers to a degree of complementarity that is at least 60%, at least 65%, at least 70%, at least 75%, at least 80%, at least 85%, at least 90%, at least 95%, at least 97%, at least 98%, or at least 99%, over a region of, for example, 8, 9, 10, 11, 12, 13, 14, 15, 16, 17, 18, 19, 20, 21, 22, 23, 24, 25, 30, 35, 40, 45, 50, or more consecutive nucleotides, or refers to two nucleic acids that hybridize under stringent conditions.

The terms “hybridization” and “hybridizing” refer to a process in which completely, substantially, or partially complementary nucleic acid strands come together under specified hybridization conditions to form a double-stranded structure or region in which the two constituent strands are joined by hydrogen bonds. Unless otherwise stated, the hybridization conditions are naturally occurring or lab designed conditions. Although hydrogen bonds typically form between adenine and thymine or uracil (A and T or U) or between cytidine and guanine (C and G), other base pairs may form (see e.g., Adams et al., The Biochemistry of the Nucleic Acids, 11th ed., 1992).

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Preferably, a nucleotide comprises a cytosine, uracil, thymine, adenine, or guanine moiety. Further, the term nucleotide also includes those species that have a detectable label, such as for example a radioactive or fluorescent moiety, or mass label attached to the nucleotide. The term nucleotide also includes what are known in the art as universal bases. By way of example, universal bases include but are not limited to 3-nitropyrrole, 5-nitroindole, or nebularine. Nucleotide analogs are, for example, meant to include nucleotides with bases such as inosine, queuosine, xanthine, sugars such as 2′-methyl ribose, and non-natural phosphodiester internucleotide linkages such as methylphosphonates, phosphorothioates, phosphoroacetates and peptides.

The terms “subject” and “patient” are used interchangeably herein to refer to an organism. e.g., a vertebrate, preferably a mammal, more preferably a human Mammals include, but are not limited to, murines, simians, humans, farm animals, sport animals, and pets such as dogs and cats. The tissues, cells and their progeny of an organism or other biological entity obtained in vivo or cultured in vitro are also encompassed within the terms subject and patient. Additionally, in some embodiments, a subject may be an invertebrate animal, for example, an insect or a nematode; while in others, a subject may be a plant or a fungus.

As used herein, “treatment,” “treating,” “palliating,” and “ameliorating” are used interchangeably. These terms refer to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the complexes of the present invention may be administered to a subject, or a subject's cells or tissues, or those of another subject extracorporeally before re-administration, at risk of developing a particular disease, condition, or symptom, or to a subject reporting one or more of the physiological symptoms of a disease, even though the disease, condition, or symptom might not have yet been manifested.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 20” may mean from 18-22. Other meanings of “about” may be apparent from the context, such as rounding off; for example “about 1” may also mean from 0.5 to 1.4.

### Discussion

According to a first embodiment, the present invention comprises a gRNA-ligand binding complex that contains both a gRNA and a ligand binding moiety. This complex has the ability to retain association with a Type V Cas protein. Within the gRNA-ligand binding complex, the gRNA may be covalently bound directly to the ligand binding moiety or bound to the ligand binding moiety through a linker.

### gRNA

The gRNA of the gRNA-ligand binding complex is either a single strand of nucleotides that has at least one region that is self-complementary or two strands of nucleotides each of which has at least one region that is complementary to a region of the other strand. Within the gRNA, regardless of whether it is a single strand of nucleotides or two strands of nucleotides, there may be one or more loops. The gRNA comprises two parts: a tracrRNA and a crRNA.

The nucleotides within the gRNA may be entirely RNA or a combination of ribonucleotides and other nucleotides such as deoxyribonucleotides. Each nucleotide may be unmodified, or one or more nucleotides may be modified, e.g., with one of the following modifications: 2′-O-methyl, 2′ fluoro or 2-aminopurine. In some embodiments over one or more ranges of one to forty or two to twenty or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, or 36 nucleotides, there are consecutively modified nucleotides or a modification pattern of every second, or every third or every fourth nucleotide being modified at its 2′ position with all other nucleotides being unmodified. Additionally or alternatively, between one or more pairs or every pair of consecutive nucleotides, there may be modified or unmodified internucleotide linkages.

In some embodiments, the crRNA is 35 to 60 nucleotides or 36 to 60 nucleotides long or 40 to 55 nucleotides long. Within the crRNA sequence are a Cas association region, which also may be referred to as a repeat region, that may be 18 to nucleotides long or 20 to 25 nucleotides long and a targeting region, which also may be referred to as a spacer region, that may be 18 to 30 nucleotides long or 20 to nucleotides long.

The targeting region contains the targeting sequence, which is a variable sequence that may be selected based on where one wishes for the Cas protein and/or effector to cause base editing. Thus, the targeting region may be designed to include a region that is complementary and capable of hybridization to a pre-selected target site of interest. For example, the region of complementarity between the targeting region and the corresponding target site sequence may be about 10, 11, 12, 13, 14, 15, 16, 17, 18, 19, 20, 21, 22, 23, 24, 25, or more than 25 consecutive nucleotides in length or it may be at least 80%, at least 85%, at least 90%, or at least 95% complementary to a region of DNA over 10, 11, 12, 13, 14, 15, 16, 17, 18, 19, 20, 21, 22, 23, 24, 25, or more than 25 consecutive nucleotides. The targeting region is a region that does not hybridize with the tracrRNA and it may be downstream of the Cas association region. The Cas association is designed based on the RNA binding domain of a Cas protein with which it is intended to associate. (Not all nucleotides within the Cas association need directly associate with the Cas protein.)

The tracrRNA sequence, may, for example, be 30 to 210 nucleotides long or 45 to 120 nucleotides long or 60 to 100 nucleotides long or 70 to 90 nucleotides long. The tracrRNA sequence comprises an anti-repeat region and a distal region. In some embodiments, the anti-repeat region is 18 to 60 nucleotides long or 25 to 50 nucleotides long or 30 to 40 nucleotides long. The distal region may, for example, be 18 to 60 nucleotides long or 25 to 50 nucleotides long or 30 to 40. The distal region is a region that does not hybridize with the crRNA and it may be upstream of the anti-repeat region.

The anti-repeat region is at least 80%, at least 85%, at least 90%, at least 95%, or 100% complementary to the Cas association region over at least 18 consecutive nucleotides of the Cas association region, and consequently, the Cas association region and the anti-repeat region are capable of hybridizing to form a hybridization region. When the tracrRNA and the crRNA hybridize over the hybridization region, the gRNA is capable of retaining association with an RNA binding domain of a Type V Cas protein. Preferably, this association is possible under both naturally occurring conditions and under laboratory conditions in which the complex is to be used.

If the tracrRNA and crRNA are part of a contiguous strand of nucleotides, there may be a loop region between the tracrRNA and the crRNA of for example, 4 to or 8 to 15 nucleotides. In 5′ to 3′ direction, the gRNA may comprise, consist essentially of or consist of the distal region, the anti-repeat region, the loop, the Cas association region and the targeting region.

Examples of tracrRNAs and crRNAs that may be of use in connection with the present invention appear below (all in 5′→3′ direction with N being any nucleotide, e.g., A, C, G, or U). In each sequence below a specific number of N nucleotides are shown. However, in any of these sequences, the N may be 16-30, which means that there can be 16, 17, 18, 19, 20, 21, 22, 23, 24, 25, 26, 27, 28, 29, or 30 nucleotides

Alicyclobacillus acidoterrestris, AacCas12b crRNA
SEQ ID NO: 1
5′-GUCGGAUCACUGAGCGAGCGAUCUGAGAAGUGGCACNNNNNNNNNNN
NNNNNNNNN-3′
SEQ ID NO: 2
5′-CGAGCGAUCUGAGAAGUGGCACNNNNNNNNNNNNNNNNNNNN-3′
SEQ ID NO: 3
5′-CUGAGAAGUGGCACNNNNNNNNNNNNNNNNNNNN-3′
AacCas12b tracrRNA
SEQ ID NO: 4
5′-GUCUAGAGGACAGAAUUUUUCAACGGGUGUGCCAAUGGCCACUUUCC
AGGUGGCAAAGCCCGUUGAGCUUCUCAAAAAG-3′
SEQ ID NO: 5
5′-GUCUAGAGGACAGAAUUUUUCAACGGGUGUGCCAAUGGCCACUUUCC
AGGUGGCAAAGCCCGUUGAGCUUCUCAAAAA-3′
SEQ ID NO: 6
5′-GUCUAGAGGACAGAAUUUUUCAACGGGUGUGCCAAUGGCCACUUUCC
AGGUGGCAAAGCCCGUUGAG-3′
AacCas12b gRNA
SEQ ID NO: 7
5′-GUCUAGAGGACAGAAUUUUUCAACGGGUGUGCCAAUGGCCACUUUCC
AGGUGGCAAAGCCCGUUGAGCUUCUCAAAUCUGAGAAGUGGCACNNNNNN
NNNNNNNNNNNNNN-3′
AaCas12b artificial chimeric gRNA (artgRNA13)
SEQ ID NO: 8
5′-GUCGUCUAUAGGACGGCGAGGACAACGGGAGUGCAGUGCUCUUUCCA
AGAGCAAACACCCCGUUGGCUUCAAGAGAAGUGGCACNNNNNNNNNNNNN
NNNNNNN-3′
Bacillus thermoamylovorans, Bth Cas12b tracrRNA
SEQ ID NO: 9
5′-CGAGGUUCUGUCUUUUGGUCAGGACAACCGUCUAGCUAUAAGUGCUG
CAGGGGUGUGAGAAACUCCUAUUGCUGGACGAUGUCUCUUUUAU-3′
Bacillus thermoamylovorans. Bth Cas12b crRNA
SEQ ID NO: 10
5′-GUCCAAGAAAAAAGAAAUGAUACGAGGCAUUAGCACNNNNNNNNNNN
NNNNNNNNN-3′
SEQ ID NO: 11
5′-AAAUGAUACGAGGCAUUAGCACNNNNNNNNNNNNNNNNNNNN-3′
SEQ ID NO: 12
5′-CGAGGCAUUAGCACNNNNNNNNNNNNNNNNNNNN-3′
Cas12e, CasX from Deltaproteobacteria
Cas12e, DpbCasX, crRNA
SEQ ID NO: 13
5′-CCGAUAAGUAAAACGCAUCAAAGNNNNNNNNNNNNNNNNNNNNN-3′
Cas12e, DpbCasX, tracrRNA
SEQ ID NO: 14
5′-GGCGCGUUUAUUCCAUUACUUUGGAGCCAGUCCCAGCGACUAUGUCG
UAUGGACGAAGCGCUUAUUUAUCGGAGA-3′
Cas12e, DpbCasX, gRNA (crRNA + tracrRNA fusion,
shorter variant)
SEQ ID NO: 15
5′-GGCGCGUUUAUUCCAUUACUUUGGAGCCAGUCCCAGCGACUAUGUCG
UAUGGACGAAGCGCUUAUUUAUCGGAGAGAAACCGAUAAGUAAAACGCAU
CAAANNNNNNNNNNNNNNNNNNNNN-3′
Cas12e, DpbCasX, gRNA (crRNA + tracrRNA fusion,
longer variant)
SEQ ID NO: 16
5′-ACAUCUGGCGCGUUUAUUCCAUUACUUUGGAGCCAGUCCCAGCGACU
AUGUCGUAUGGACGAAGCGCUUAUUUAUCGGAGAGAAACCGAUAAGUAAA
ACGCAUCAAAGNNNNNNNNNNNNNNNNNNNN-3′
Cas12f nucleases
Un1Cas12f1 (Cas14a1) gRNA
SEQ ID NO: 17
5′-GGGCUUCACUGAUAAAGUGGAGAACCGCUUCACCAAAAGCUGUCCCU
UAGGGGAUUAGAACUUGAGUGAAGGUGGGCUGCUUGCAUCAGCCUAAUGU
CGAGAAGUGCUUUCUUCGGAAAGUAACCCUCGAAACAAAUUCAUUUUUCC
UCUCCAAUUCUGCACAAGAAAGUUGCAGAACCCGAAUAGACGAAUGAAGG
AAUGCAACNNNNNNNNNNNNNNNNNNNN-3′
Cas12f tracrRNA
SEQ ID NO: 18
5′-CUUCACCAAAAGCUGUCCCUUAGGGGAUUAGAACUUGAGUGAAGGUG
GGCUGCUUGCAUCAGCCUAAUGUCGAGAAGUGCUUUCUUCGGAAAGUAAC
CCUCGAAACAAAUUCAUUU-3′
Cas12f crRNA
SEQ ID NO: 19
5′-GAAUGAAGGAAUGCAACNNNNNNNNNNNNNNNNNNNN-3′
SEQ ID NO: 20
5′GUUGCAGAACCCGAAUAGACGAAUGAAGGAAUGCAACNNNNNNNNNNN
NNNNNNNNN-3′
Cas12f gRNA (crRNA + tracrRNA fusion)
SEQ ID NO: 21
5′-CUUCACCAAAAGCUGUCCCUUAGGGGAUUAGAACUUGAGUGAAGGUG
GGCUGCUUGCAUCAGCCUAAUGUCGAGAAGUGCUUUCUUCGGAAAGUAAC
CCUCGAAACAAAUUCAUUUGAAAGAAUGAAGGAAUGCAACNNNNNNNNNN
NNNNNNNNNN-3′

### Ligand Binding Moiety

The ligand binding moiety is an element that is capable of reversibly associating with a ligand by for example, forming non-covalent interactions. In some embodiments, the ligand binding moiety is an aptamer. The ligand binding moiety may be bound to the gRNA directly, e.g., through a covalent bond, or through a linker. The association of the ligand binding moiety with the gRNA, regardless of whether directly through a covalent bond or through a linker, may be at any of a number of locations, for example, the tracrRNA in either the anti-repeat region or the distal region or in the crRNA in the targeting region or the Cas association region, or a loop between the tracrRNA and the crRNA, if present. A ligand binding moiety is bound directly to a gRNA if it is bound to a nucleotide within the gRNA, e.g., to the backbone phosphate of a unit or to a sugar moiety or to a nitrogenous base of a nucleotide.

By way of non-limiting examples, the ligand binding moiety may be bound directly (through e.g., a covalent bond) to the 3′ end of the gRNA or to the 5′ end of the gRNA if the gRNA is a single strand (which correspond to the 5′ end of the tracrRNA and the 3′ end of the crRNA) or to the 3′ end of the crRNA, the 3′ end of the tracrRNA, the 5′ end of the tracrRNA or the 5′ end of the crRNA if they are separate strands. Thus, the ligand binding moiety may be bound to the first or last nucleotide in the gRNA or in either strand of the gRNA. Alternatively, the ligand binding moiety may be bound to a nucleotide that is not the first or last nucleotide in the gRNA (if a single stranded) or tracrRNA or crRNA (if separate strands).

When the ligand binding moiety is a nucleotide sequence, and it is bound directly to the 5′ end or the 3′ end of the gRNA, it may be in the same 5′ to 3′ orientation as the gRNA. In these circumstances, there is a continuous strand of nucleotides that contains both the ligand binding moiety and the gRNA and thus is either
5′—[gRNA]-[ligand binding moiety]-3′ or
5′—[ligand binding moiety]-[gRNA]-3′.
   In other embodiments the ligand binding moiety may be directly attached to the gRNA in an opposite orientation and thus is either
5′—[gRNA]-3′-3′-[ligand binding moiety]-5′ or
3′—[ligand binding moiety]-5′-5′-[gRNA]-3′.

The ligand binding moiety may also be attached to the gRNA at a position other than the 5′ end or the 3′. When the ligand binding moiety is a nucleotide sequence it may be inserted in the gRNA, and thus there may, for example, be a first section of the gRNA that is 5′ of the ligand binding moiety and a second section of the gRNA that is 3′ of the ligand binding moiety such that there is one oligonucleotide sequence” 5′-[first section of gRNA]-[ligand binding moiety]-[second section of gRNA]-3′. Relative to a gRNA that does not contain the ligand binding moiety, the complex that contains the gRNA and the ligand binding moiety inserted there may be no deletion of nucleotides from either the Cas association region or the targeting region. Alternatively, there may be a deletion of one or more nucleotide (e.g., 1 to 10 nucleotides) at the location of insertion.

In some embodiments, the ligand binding moiety forms a stem and loop complex. In some embodiments, this stem and loop complex of the ligand binding moiety may be at a location at which in the absence of the ligand binding moiety there is a bulge or another stem-loop complex.

In some embodiments, when the ligand binding moiety is not a nucleotide sequence and it is bound to the gRNA as the location other than the 5′ or 3′ end it may, for example, be bound between two consecutive nucleotides:
5′-[first section of gRNA]-[ligand binding moiety]-[second section of gRNA]-3′,
   or it may be attached at either the 5′ or 3′ end of the gRNA to the phosphorous moiety, the sugar at e.g., the 2′, 3′ or 5′ position or the nitrogenous base. These ligand binding moieties may, for example, be bound at the location of a bulge or a stem loop of the gRNA or at a location that has an absence of a bulge or stem-loop.

In other embodiments, the ligand binding moiety is bound to a linker that is bound to the to the 3′ end of the gRNA or to the 5′ end of the gRNA or to another one or more locations within the gRNA. In some embodiments, each of the linker and the ligand binding moiety may independently comprise, consist essentially, or consist of nucleotides. In some embodiments, each of the linker and the ligand binding moiety may independently comprise, consist essentially, or consist of a moiety other than nucleotides.

When the ligand binding moiety is a nucleotide sequence and it is bound through linker to the 5′ end or the 3′ end of the gRNA each of the gRNA, the linker and ligand binding moiety may be in the same 5′ to 3′ orientation. In these circumstances, there is a continuous strand of nucleotides that contains both the ligand binding moiety and the gRNA either
5′-[gRNA]-[linker]-[ligand binding moiety]-3′ or
5′-[ligand binding moiety]-[linker]-[gRNA]-3′.
   In other embodiments the ligand binding moiety and/or the linker can be directly attached to the gRNA in an opposite orientation and thus is
5′-[gRNA]-3′-3′-[linker]-5′-3′-[ligand binding moiety]-5′ or
5′-[gRNA]-3′-5′-[linker]-3′-3′-[ligand binding moiety]-5′ or
3′-[ligand binding moiety]-5′-3′-[linker]-5′-5′-[gRNA]-3′ or
3′-[ligand binding moiety]-5′-5′-[linker]-3′-5′-[gRNA]-3′.

The ligand binding moiety may also be attached to the gRNA through a linker at a position other than the 5′ end or the 3′. When the ligand binding moiety and the linker are nucleotide sequences, they may be inserted in the gRNA, and thus there may for example be a first section of the gRNA that is 5′ of the ligand binding moiety and linker and a second section of the gRNA that is 3′ of the ligand binding moiety and linker. In some embodiments, there are two linkers that flank the ligand binding moiety.

When there is only one linker sequence it may be either 5′ or 3′ of the ligand binding moiety such that the complex is
5′-[first section of gRNA]-[linker]-[ligand binding moiety]-[second section of gRNA]-3′, or
5′-[first section of gRNA]-[ligand binding moiety]-[linker]-second section of gRNA]-3′.

When there is are two linker sequences a first linker may be 5′ of the ligand binding moiety and the second linker is 3′ of the ligand binding moiety, with such that the complex is
5′-[first section of gRNA]-[first linker]-[ligand binding moiety]-[second linker]-[second section of gRNA]-3′.

In some embodiments, each of the first section of gRNA the linker, the ligand binding moiety, the second linker an the second section of gRNA are nucleotide sequences in the same orientation. In other embodiments, one or more of the first linker, ligand binding moiety and the second linker are in the opposite orientation to that of the first section of gRNA and the second section of gRNA, which are in the same orientation.

In some embodiments, when the ligand binding moiety is between the first section of the gRNA and the second section of the gRNA (and if one or two linkers are present they are also between the first section of the gRNA and the second section of the gRNA):
the first section of the gRNA contains a portion of the distal region, and the second section of the gRNA contains the remainder of the distal region and the entire anti-repeat region;
the first section of the gRNA contains a portion of the distal region, and the second section of the gRNA contains the remainder of distal region, the entire anti-repeat region and the crRNA;
the first section of the gRNA contains the entire the distal region, and the second section of the gRNA contains the entire anti-repeat region;
the first section of the gRNA contains the entire the distal region, and the second section of the gRNA contains the entire anti-repeat region and the crRNA;
the first section of the gRNA contains the entire distal region and a portion of the anti-repeat region, and the second section of the gRNA contains the remainder of the anti-repeat region;
the first section of the gRNA contains the entire distal region and a portion of the anti-repeat region, and the second section of the gRNA contains the remainder of the anti-repeat region and the crRNA;
the first section of the gRNA contains the tracrRNA and the second section of the gRNA contains the crRNA;
the first section of the gRNA contains the tracrRNA and a portion of the Cas association region, and the second section of the gRNA contains the remainder of the Cas association region and the entire targeting region;
the first section of the gRNA contains a portion of the Cas association region and the second section of the gRNA contains the remainder of the Cas association region and the entire targeting region;
the first section of the gRNA contains the tracrRNA and the entire Cas association region and the second section of the gRNA contains the entire targeting region;
the first section of the gRNA contains the entire Cas association region and the second section of the gRNA contains the entire targeting region;
the first section of the gRNA contains the tracrRNA, the entire Cas association region and a portion of the targeting region, and the second section of the gRNA contains the remainder of the targeting region; and
the first section of the gRNA contains the entire Cas association region and a portion of the targeting region and the second section of the gRNA contains the remainder of targeting region.
   Relative to a gRNA that does not contain the ligand binding moiety (and optionally one or more linkers), in a complex that contains the gRNA and the ligand binding moiety inserted therein, there may be no deletion of nucleotides from or around the region of insertion. Alternatively, there may be a deletion of one or more nucleotides (e.g., 1 to 10 nucleotides) at or around the location of insertion.

When there are two linkers present, they may be of sufficient complementary such that they can hybridize to each under. For example, each linker may be 1 to 20 nucleotides long and the linkers may be at least 80%, at least 85%, at least 90%, at least 95% at least 98% or 100% complementary and have no bulges one or more bulges. In some embodiments, the linkers are the same size; in other embodiments, the linkers are different sizes.

When a ligand binding moiety is bound to the loop of the gRNA, either directly or through a linker, the bonding may, for example, be at the first nucleotide in the loop, the second nucleotide in the loop, the third nucleotide in the loop, the fourth nucleotide in the loop, the center nucleotide in the loop if the loop has an odd number of nucleotides or one of the two center most nucleotides in the loop if the loop has an even number of nucleotides, or the last nucleotide in the loop. Any one or more of the aforementioned nucleotides and/or the 5′ and/or 3′ internucleotide linkage corresponding to them may be modified. These modifications may, for example, occur where the ligand binding moiety is bound to the gRNA (directly or through a linker) or only at locations other than where the ligand binding moiety is bound to the gRNA (directly or through a linker). For example, the ligand binding moiety can be attached to a 2′ position of a sugar or attached to a nitrogenous base in the gRNA oligonucleotide sequence.

In some embodiments, the ligand binding moiety comprises, consists essentially of, or consists of an oligonucleotide sequence that is unmodified or comprises one or more modified nucleotides. For example, the ligand binding moiety may be 10 to 50 or 18 to 50 nucleotides long. In some embodiments the ligand binding moiety forms a stem-loop structure. If there is no linker present, the ligand binding moiety may appear as an extension of the gRNA sequence immediately 5′ or 3′ of the gRNA or 5′ or 3′ of the tracrRNA or the crRNA or it may appears as an insertion.

In some embodiments, the ligand binding moiety comprises, consists essentially of, or consists of biotin or streptavidin.

In some embodiments, the ligand binding moiety is selected from the group consisting of moieties that associate with the following ligands: MS2 coat protein (MCP), Ku, PP7 coat protein (PCP), Com RNA binding protein or the binding domain thereof, SfMu, Sm7, Tat, Glutathione S-transferase (GST), CSY4, Qbeta, COM, pumilio, Anti-His Tag (6H7), SNAP-Tag, lambdaN22, a lectin (in which case ligand binding moiety may be carbohydrate or glycan or oligosaccharide), and PDGF beta-chain. In some embodiments, the ligand binding moiety is an aptamer that comprises deoxyribonucleotides, ribonucleotides or a combination of both. Therefore, as non-limiting examples, one may use DNA aptamers, RNA aptamers, DNA aptamers with modified nucleosides in the backbones, RNA aptamers with modified nucleosides in the backbones and combinations thereof.

In some embodiments, a naturally occurring MS2 aptamer is used as the ligand binding moiety. In other embodiments, one uses an MS2 C-5 mutant or an MS2 F-5 mutant or a modified MS2, e.g., MS2 in which there is one or more, e.g., 1, 2, 3, 4, 5, 6, 7, 8, 9, 10, 11, or 12, modified nucleotides such as an amino purine, at position 10, wherein position 10 is the tenth nucleotide from the 5′ end of an aptamer. The 2-amino purine may, for example, be 2-amino purine is 2′ deoxy-2-aminopurine or 2′ ribose 2-aminopurine. The modification at any one position may be in addition to a modification at another position or to the exclusion of a modification at any or all of the other positions.

In some embodiments, the ligand binding moiety is an aptamer that comprises a 5′ modified nucleotide, wherein the 5′ modified nucleotide comprises at least one of a 2′ modification, a 5′ PO₄ group, or a modification of the nitrogenous base.

In some embodiments, the ligand binding moiety is an aptamer that is or comprises one part of an aptamer-ligand pair, and as discussed below, the effector is linked to or comprises the other part of the aptamer-ligand pair. For example, the aptamer may comprise a MS2 operator motif that specifically binds to an MS2 coat protein, MCP. As persons of ordinary skill in the art will appreciate alternatively, the aptamer can comprise the MCP moiety (or other ligand) in which case the effector would comprise or be linked to the MS2 operator motif (or other corresponding ligand binding moiety).

### Linkers

A linker, when present, may be a species that connects the ligand binding moiety to the gRNA. It may be attached to each of the ligand binding moiety and the gRNA at one location or it may be attached to either or both of the gRNA and the ligand binding moiety at a plurality of locations. Attachments at a plurality of locations may allow for greater control in three dimensional space of the ligand binding moiety and in turn the effector to be used.

By way of non-limiting examples, the linker may attach to the gRNA at one location and to the ligand binding moiety at two or more locations; or the linker may attach to the ligand binding moiety at one location and to the gRNA at two or more locations. When the linker is attached to the gRNA at two or more locations, the linker may be attached to the gRNA exclusively in the targeting region, exclusively in the Cas association region or in both the targeting region and the Cas association region, exclusively in the anti-repeat region, exclusively in the distal region, in both the anti-repeat region and the distal region, in both the distal region and the targeting region, in both the anti-repeat region and the Cas association region, in both the anti-repeat region and the targeting region, or in both the Cas association region and the distal region.

In some embodiments, the linker comprises, consists essentially of, or consists of an oligonucleotide sequence and optionally the linker comprises at least one or a plurality of 2′ modifications, e.g., all nucleotides are 2′ modified nucleotides within the linker. The nucleotide sequence may be random or intentionally designed not to be undesirably complementary to sequence within the aptamer, the gRNA or the target site of the DNA.

In some embodiments, the linker comprises, consists essentially of, or consists of at least one phosphorothioate linkage.

In some embodiments, the linker comprises, consists essentially of, or consists of a levulinyl moiety.

In some embodiments, the linker comprises, consists essentially of, or consists of an ethylene glycol moiety.

In some embodiments, the linker comprises or is selected from the group consisting of 18S, 9S or C3.

In some embodiments, the linker is a nucleotide sequence that is one to sixty or one to twenty-four or two to twenty or five to fifteen nucleotides long.

Additionally, in some embodiments, the linker is GC rich, e.g., having at least 50%, at least 60%, at least 70%, at least 80% or at least 90% GC nucleotides. When a linker comprises nucleotides, it may, for example, be single stranded or double stranded or partially single stranded and partially double stranded. Additionally, when a linker is an oligonucleotide, the linker may be exclusively RNA, exclusively DNA or a combination thereof.

In some embodiments, the linker is a nucleotide sequence that is upstream or downstream of the ligand binding moiety. When the linker is upstream of a ligand binding moiety and the gRNA is upstream of the linker, there may be another sequence that is complementary to the linker that is downstream of the ligand binding moiety. Similarly, when the linker is downstream of a ligand binding moiety and the gRNA is downstream of the linker, there may be another sequence that is complementary to the linker that is upstream of the ligand binding moiety. As persons of ordinary skill in the art will recognize, complementarity is determined when the oligonucleotide self-folds and the strands align with each relevant section in a 5′ to 3′ direction.

Thus, in some embodiments, the ligand binding moiety, e.g., MS2 has an upstream sequence that is 1 to 12 nucleotides long and a downstream sequence that is 1 to 12 nucleotides long, wherein the upstream and downstream sequences immediately flank the ligand binding moiety (i.e., there are no other nucleotides between the ligand binding moiety and each of the upstream and downstream sequences) and the upstream sequence is complementary to the downstream sequence. In some embodiments, each of the upstream sequence and the downstream sequence is 1 nucleotide long, 2 nucleotides long, 3 nucleotides long, 4 nucleotides long, 5 nucleotides long, 6 nucleotides long, 7 nucleotides long, 8 nucleotides long, 9 nucleotides long, 10 nucleotides long, 11 nucleotides long, or 12 nucleotides long. In one embodiment each of the upstream sequence and the downstream sequence comprises or is GC. When there are both upstream and downstream sequences, they may also be referred to as extension sequences.

### Modifications

In some embodiments, at least one of the gRNA or the ligand binding moiety is modified, or if a linker is present, at least one of the gRNA, the ligand binding moiety or the linker is modified. The modification refers to the introduction of a moiety or species that does not occur under naturally occurring conditions. Modifications may be used to increase one or both of stability and specificity. In some embodiments, stability is improved with respect to resistance to one or both of the active domain of the Cas protein (e.g., RuvC domain) and the active domain of one or more other enzymes within the system into which a complex of the present invention is introduced, including but not limited to any effector. Specificity is improved when a modification reduces the likelihood of an off-target effect and/or increases the likelihood that a base editing complex of the present invention will reach its target site. Nucleotides may be modified at the ribose, phosphate linkage, and/or base moiety. For example, a phosphorothioate backbone may be used, at one, a plurality or all positions within the gRNA, the anti-repeat region, the distal region, the targeting region or the Cas association region and/or the ligand binding moiety and/or linker if present.

In some embodiments, the modification is the presence of one or more 2′ modified nucleotides (e.g., 2′-O-methyl or 2′-fluoro) and/or the presence of a phosphorothioate internucleotide linkage or the introduction of a 5′-PO₄ group of the gRNA and/or ligand binding moiety.

In some embodiments, a modification or set of modifications is selected such that it imparts resistance to a RuvC active nuclease domain relative to a gRNA-ligand binding complex that lacks that modification or set of modifications. The resistance may, in some embodiments, be caused by steric hindrance. In some embodiments, the modification(s) is/are located within and/or between one or more if not all of the nucleotides within the targeting region.

When more than one modification is present, the modifications may, for example, all be in the targeting region; all be in the Cas association region; all be in the anti-repeat region; all be in the distal region; all be in the ligand binding moiety; all be in the linker if present; be in both the targeting region and the Cas association region; be in both the Cas association region and the ligand binding moiety; be in both the Cas association region and the linker if present; be in both the targeting region and the ligand binding moiety; be in both the targeting region and the linker if present; be in both the ligand binding moiety and the linker if present; be in all three of the Cas association region, the targeting region and the ligand binding moiety; be in the Cas association region, the targeting region and the linker if present; be in the Cas association region, the ligand binding moiety and the linker if present; be in the targeting region, the ligand binding moiety and the linker if present; be in each of the Cas association region, the targeting region, the ligand binding moiety and the linker if present; be in the anti-repeat region and the distal region; be in the anti-repeat region and the Cas association region; be in the anti-repeat region and the targeting region; be in the distal region and the Cas association region; be in the distal region and targeting region; be in all regions of the gRNA except the distal region; be in all regions of the gRNA except the anti-repeat region; be in all regions of the gRNA except the Cas association region; be in all regions of the gRNA except the targeting region; be in each of the targeting region, the Cas association region, the anti-repeat region and the distal region; be in the ligand binding moiety and all regions of the gRNA except the anti-repeat region; be in the ligand binding moiety and all regions of the gRNA except the Cas association region; be in the ligand binding moiety and in all regions of the gRNA except the targeting region; and be in the ligand binding moiety and each of the targeting region, the Cas association region, the anti-repeat region, and the distal region.

In some embodiments, there are one to sixty or one to thirty or one to ten or ten to twenty or twenty to thirty or thirty to forty or forty to fifty or fifty to sixty 2′ modifications. By way of non-limiting examples, the set of 2′ modifications may be located: in the targeting region; in the anti-repeat region; in the distal region; in the ligand binding moiety if the ligand binding moiety is or comprises an oligonucleotide sequence; or in the Cas association region or in combinations thereof. The modifications may be on consecutive nucleotides or there may be one or more pairs of unmodified nucleotides between modified nucleotides in regular or irregular patterns. By way of a further non-limiting example, within a gRNA any one or more of positions 1, 2, 3, 4, 5, 6, 7, 8, 9, 10, 11, 12, 13, 14, 15, 16, 17, or 18 comprises a 2′-O-alkyl group, wherein the positions are measured from the 5′ end or the 3′ end of the gRNA or the tracrRNA or the crRNA.

In some embodiments, in addition to or in the absence of 2′ modified nucleotides there are modified internucleotide linkages such as phosphorothioate linkage. Examples of modifications to the backbones of the gRNA, the ligand binding moiety (in an oligonucleotide), and the linker (if present and an oligonucleotide), include but are not limited to phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e., a single inverted nucleoside residue that may be a basic (the nucleobase is missing or has a hydroxyl group in place thereof). Various salts (such as, for example, potassium or sodium), mixed salts and free acid forms of the aforementioned internucleotide linkages are also included within the scope of the present invention.

Also within the scope of the present invention is the use of polynucleotide backbones that do not include a phosphorus atom therein and instead have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These modifications include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments, one or more of the parts of a complex has one to sixty or one to twenty or one to ten or ten to twenty or twenty to thirty or thirty to forty to forty to fifty or fifty to sixty phosphorothioate linkages. These phosphorothioate linkages may: all be in the targeting region; all be in the Cas association region; all be in the anti-repeat region; all be in the distal region; all be in the ligand binding moiety; all be in the linker if present; be in both the targeting region and the Cas association region; be in both the Cas association region and the ligand binding moiety; be in both the Cas association region and the linker if present; be in both the targeting region and the ligand binding moiety; be in both the targeting region and the linker if present; be in both the ligand binding moiety and the linker if present; be in all three of the Cas association region, the targeting region and the ligand binding moiety; be in the Cas association region, the targeting region and the linker if present; be in the Cas association region, the ligand binding moiety and the linker if present; be in the targeting region, the ligand binding moiety and the linker if present; be in each of the Cas association region, the targeting region, the ligand binding moiety and the linker if present; be in the anti-repeat region and the distal region; be in the anti-repeat region and the Cas association region; be in the anti-repeat region and the targeting region; be in the distal region and the Cas association region; be in the distal region and targeting region; be in all regions of the gRNA except the distal region; be in all regions of the gRNA except the anti-repeat region; be in all regions of the gRNA except the Cas association region; be in all regions of the gRNA except the targeting region; be in each of the targeting region, the Cas association region, the anti-repeat region and the distal region; be in the ligand binding moiety and all regions of the gRNA except the anti-repeat region; be in the ligand binding moiety and all regions of the gRNA except the Cas association region; be in the ligand binding moiety and in all regions of the gRNA except the targeting region; and be in the ligand binding moiety and each of the targeting region, the Cas association region, the anti-repeat region, and the distal region.

Any nucleotide within a complex of the present invention may include one or more substituted sugar moieties. These nucleotides may comprise a sugar substituent group selected from: OH; H; F; O—, S—, or N-alkyl; O—, S—, or N-alkenyl; O—, S— or N-alkynyl; or O-alkyl-Co-alkyl, wherein the alkyl, alkenyl and alkynyl may be substituted or unsubstituted C1 to C10 alkyl or C2 to C10 alkenyl and alkynyl. Particularly suitable are O((CH₂)nO)mCH₃, O(CH₂)nOCH₃, O(CH₂)nNH₂, O(CH₂)nCH₃, O(CH₂)nONH₂, and O(CH₂)nON((CH₂)nCH₃)₂, where n and m are from 1 to about 10. Other suitable nucleotides comprise a sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. By way of a non-limiting example, a suitable modification includes 2′-methoxyethoxy (2′-O-CH₂CH₂OCH₃, also known as 2′-O-(2-methoxyethyl) or 2′-MOE) (Martin et al., Helv. Chim. Acta, 1995, 78, 486-504) or another alkoxyalkoxy group. A further suitable modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂CH₂CH₂NH₂), allyl (—CH₂—CH═CH₂), —O-allyl CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide.

Any nucleotide within a complex of the present invention may also include nucleobase (often referred to in the art simply as “base”) modifications or substitutions. Modified nucleobases include, but are not limited to other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include, but are not limited to tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one) and 5-methoxy uracil.

Heterocyclic base moieties may also include, but are not limited to, those in which the purine or pyrimidine base is replaced with other heterocycles, for example, 7-deaza-adenine, 7-deazaguanosine, 2-aminopyridine and 2-pyridone. Examples of other nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound: 5-substituted pyrimidines, 6-azapyrimidines and N-2, N-6 and O-6 substituted purines, including 2-aminopropyladenine, 5-propynyluracil and 5-propynylcytosine. Additionally, 5-methylcytosine substitutions may be advantageous when combined with 2′-O-methoxyethyl sugar modifications.

In some embodiments, there are two ligand binding moieties associated with a gRNA: a first ligand binding moiety and a second ligand binding moiety. Optionally, there may be two linkers: a first linker and a second linker, wherein the first ligand binding moiety is attached to the first linker and the second ligand binding moiety is attached to the second linker. In these embodiments, the first linker and the second linker may each be attached to the Cas association region; or the first linker and the second linker may each be attached to the targeting region; or one of the first linker and the second linker may be attached to the Cas association region and the other of the first linker and the second linker may be attached to the targeting region, or the first linker and the second linker may each be attached to the anti-repeat region; or the first linker and the second linker may each be attached to the distal region; or one of the first linker and the second linker may be attached to the distal region and the other of the first linker and the second linker may be attached to the anti-repeat region; or one of the first linker and the second linker may be attached to the distal region and the other of the first linker and the second linker may be attached to the Cas association region; or one of the first linker and the second linker may be attached to the distal region and the other of the first linker and the second linker may be attached to the targeting region; or one of the first linker and the second linker may be attached to the anti-repeat region and the other of the first linker and the second linker may be attached to the Cas association region; or one of the first linker and the second linker may be attached to the anti-repeat region and the other of the first linker and the second linker may be attached to the targeting region; or one of the first linker and the second linker may be attached to a loop region if present and the other of the first linker and the second linker may be attached to one of the targeting region, the Cas association region, the anti-repeat region, or the distal region.

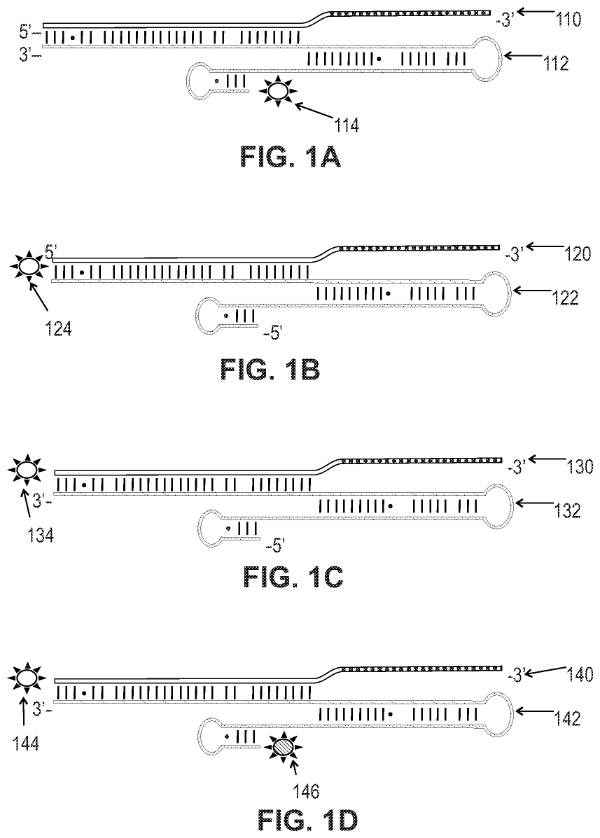

FIG. 1A is a representation of a gRNA-ligand binding complex of the present invention in which a ligand binding moiety 114 is bound to the 5′ end of a tracrRNA 112 in a Cas12b gRNA in which the crRNA 110 is a separate strand that hybridizes with part of the tracrRNA. FIG. 1B is a representation of a gRNA-ligand binding complex of the present invention in which a ligand binding moiety 124 is bound to the 3′ end of a tracrRNA 122. Also shown is the crRNA 120. FIG. 1C is a representation of a gRNA-ligand binding complex of the present invention in which a ligand binding moiety 134 is bound to the 5′ end of a crRNA 130. Also shown is the tracrRNA 132. FIG. 1D is a representation of a gRNA-ligand binding complex of the present invention in which a first ligand binding moiety 146 is bound to the 5′ end of a tracrRNA 142 and a second ligand binding moiety 144 is bound to the 5′ end of a crRNA 140. By way of non-limiting examples, each of the ligand binding moieties shown in FIGS. 1A to 1D and in all other figures may, for example, be a DNA or an RNA aptamer, a carbohydrate or oligosaccharide group, a benzylguanine or benzylcytosine group for SNAP/CLIP tagging, a bioconjugation group (e.g., azide-alkyne group for click chemistry), biotin or other functional group used for affinity or covalent binding. In a dual ligand binding moiety system such as shown in FIG. 1D, different or similar functional groups can be attached to each molecule to further increase functionality.

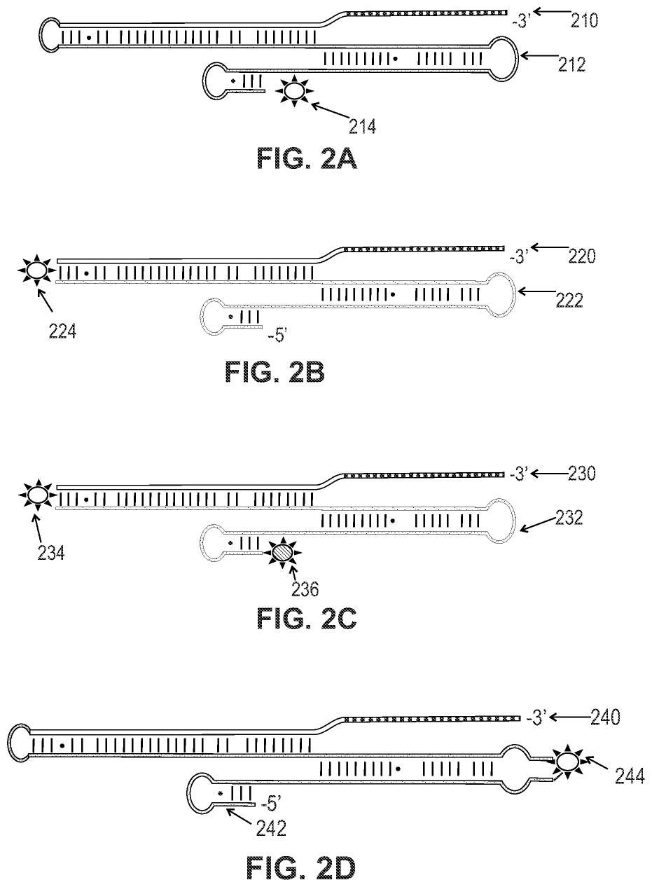

FIG. 2A is a representation of a gRNA-ligand binding complex of the present invention in which a ligand binding moiety 214 is bound to the 5′ end of a tracrRNA 212 in a Cas gRNA with the crRNA 210 being part of the same strand of nucleotides as the tracrRNA. FIG. 2B is a representation of a gRNA-ligand binding complex of the present invention in which a ligand binding moiety 224 is bound to a loop between tracrRNA 222 and crRNA 220 in a Cas12b gRNA. FIG. 2C is a representation of a gRNA-ligand binding complex of the present invention in which a first ligand binding moiety 234 is bound to the loop region between the tracrRNA 232 and the crRNA 230 and a second ligand binding moiety 236 is bound to the 5′ end of the tracrRNA in a Cas12b gRNA. FIG. 2D is a representation of a gRNA-ligand binding complex of the present invention in which a ligand binding moiety 244 is bound to a loop within the tracrRNA region 242 and a crRNA 240 forms the 3′ portion of the gRNA in a Cas12b gRNA.

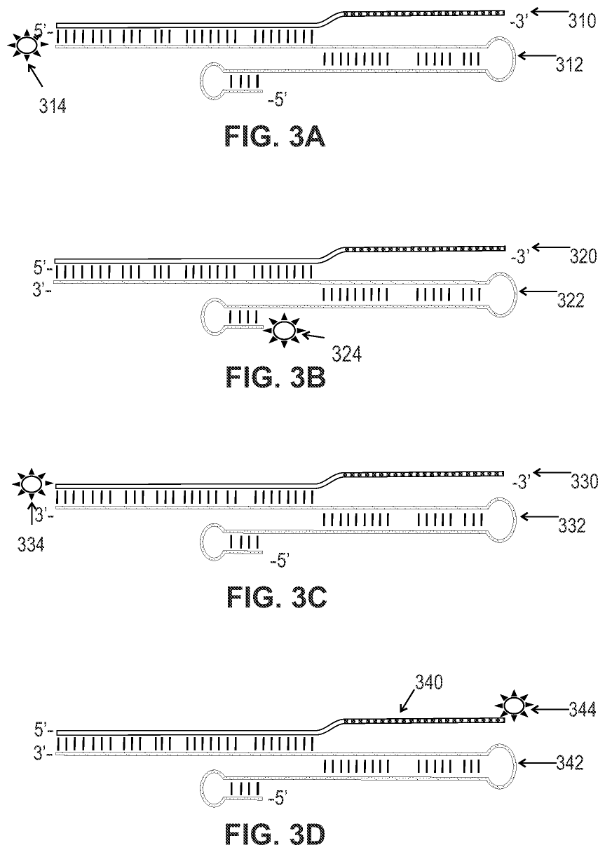

FIG. 3A is a representation of a gRNA-ligand binding complex of the present invention in which a ligand binding moiety 314 is bound to the 3′ end of a tracrRNA 312 in a Cas12e gRNA that has a separate crRNA strand 310. FIG. 3B is a representation of a gRNA-ligand binding complex of the present invention in which a ligand binding moiety 324 is bound to the 5′ end of a tracrRNA 322 in a Cas12e gRNA that has a separate crRNA strand 320. FIG. 3C is a representation of a gRNA-ligand binding complex of the present invention in which a ligand binding moiety 334 is bound to the 5′ end of a crRNA 330 in a Cas12e gRNA that has a separate tracrRNA 332. FIG. 3D is a representation of a gRNA-ligand binding complex of the present invention in which a ligand binding moiety 334 is bound to the 3′ end of a crRNA 340 in a Cas12e gRNA in which there is a separate strand that forms the tracrRNA 342.

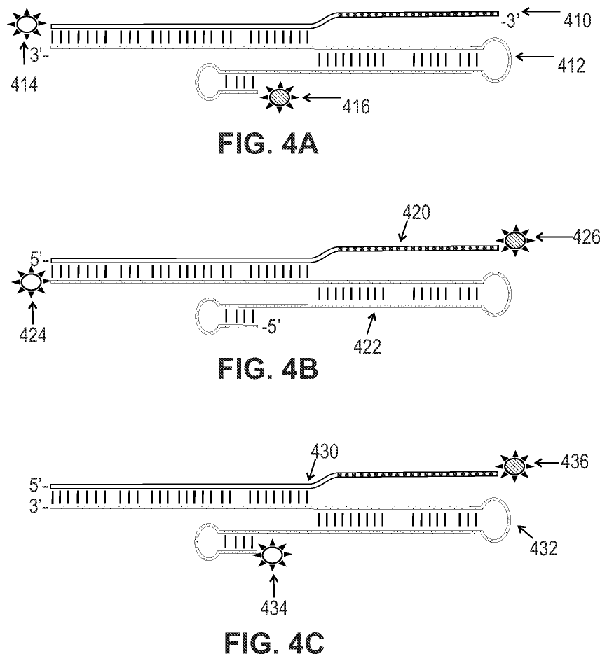

FIG. 4A is a representation of a gRNA for a Cas 12e system in which a first ligand binding moiety 414 is attached to the 5′ end of the crRNA 410 and a second ligand binding moiety 416 is attached to the 5′ end of the tracrRNA 412. FIG. 4B is a representation of a gRNA for a Cas 12e system in which a first ligand binding moiety 426 is attached to the 3′ end of the crRNA 420 and a second ligand binding moiety 424 is attached to the 3′ end of the tracrRNA 422. FIG. 4C is a representation of a gRNA for a Cas 12e system in which a first ligand binding moiety 434 is attached to the 5′ end of the tracrRNA 432 and a second ligand binding moiety 436 is attached to the 3′ end of the crRNA 430.

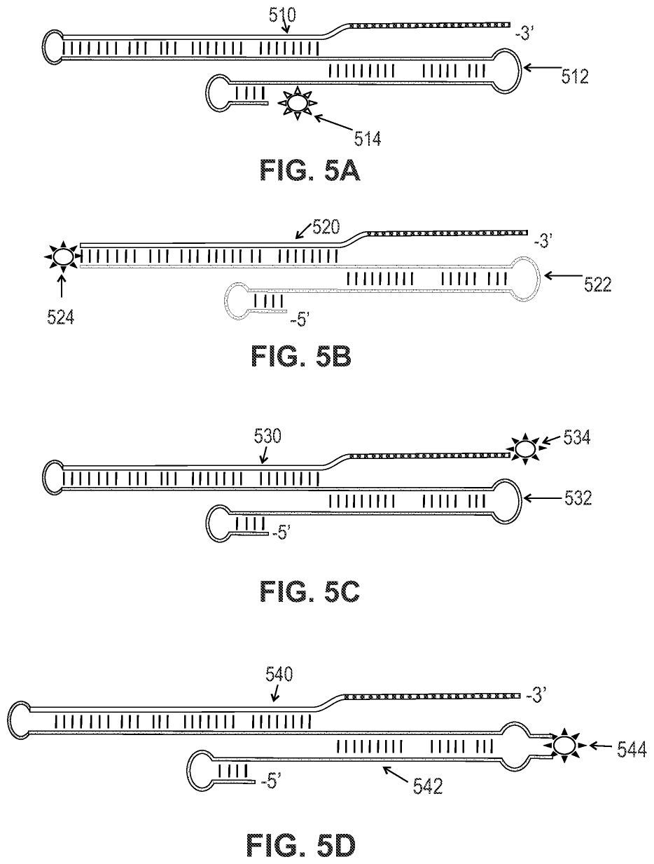

FIG. 5A is a representation of a gRNA for a Cas 12e system in which a ligand binding moiety 514 is attached to the 5′ end of the tracrRNA 512 and the crRNA 510 is part of the same strand of nucleotides as the tracrRNA. FIG. 5B is a representation of a gRNA for a Cas 12e system in which a ligand binding moiety 524 is attached to the loop between the tracrRNA 522 and the crRNA 520. FIG. 5C is a representation of a gRNA for a Cas 12e system in which a ligand binding moiety 534 is attached to the 3′ end of the crRNA 530 that is downstream of and forms a hairpin with the tracrRNA 532. FIG. 5D is a representation of a gRNA for a Cas 12e system in which a ligand binding moiety 544 is attached to a loop within a tracrRNA 542 and a crRNA 540 forms a hairpin with the tracrRNA.

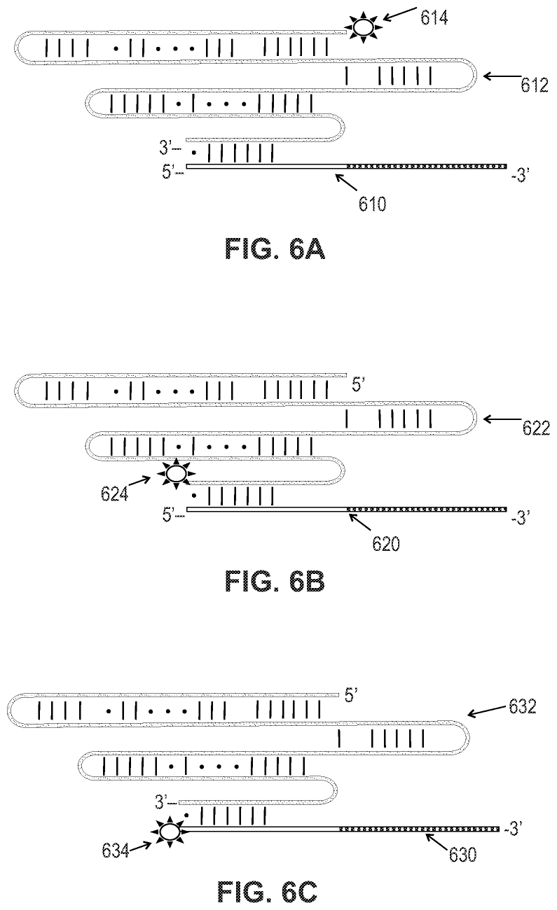

FIG. 6A is a representation of a gRNA for a Cas 12f system in which a ligand binding moiety 614 is attached to the 5′ end of the tracrRNA 612 and the crRNA 610 is a separate strand that forms a hybridization region with the tracrRNA. FIG. 6B is a representation of a gRNA for a Cas 12f system in which a ligand binding moiety 624 is attached to the 3′ end of the tracrRNA 622 and the crRNA 620 is a separate strand that forms a hybridization region with the tracrRNA. FIG. 6C is a representation of a gRNA for a Cas 12f system in which a ligand binding moiety 634 is attached to the 5′ end of the crRNA 630 and the tracrRNA 632 is shown.

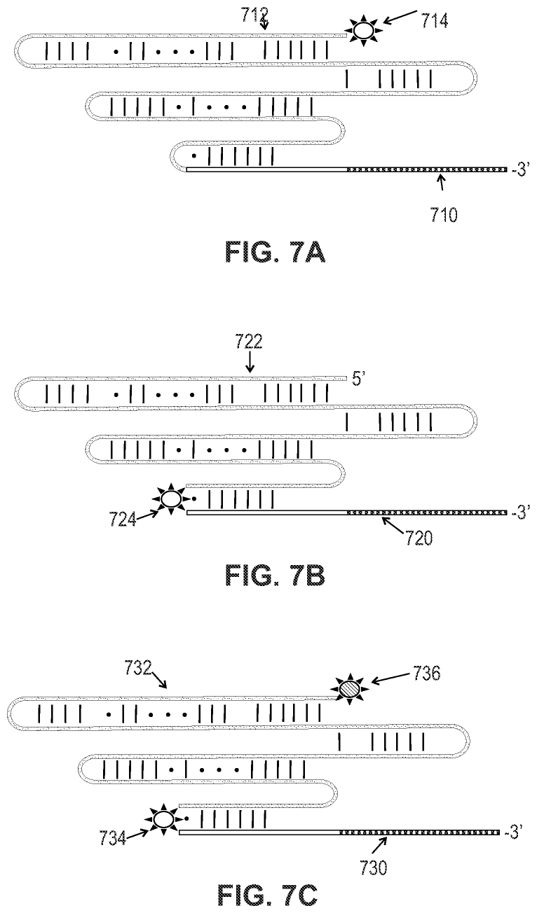

FIG. 7A shows a gRNA that is a single strand of nucleotides that contains both the tracrRNA and the crRNA for a Cas 12f system in which the ligand binding moiety 714 is bound to the 5′ end of the tracrRNA 712, which forms a contiguous strand of nucleotides with the crRNA 710. FIG. 7B shows a gRNA that is a single strand of nucleotides that contains both the tracrRNA 722 and the crRNA 720 for a Cas 12f system in which the ligand binding moiety 724 is bound to the loop between the tracrRNA and the crRNA. FIG. 7C shows a gRNA for a Cas 12f system in which a first ligand binding moiety 736 is located at the 5′ end of the tracrRNA 732 of the gRNA and a second ligand binding moiety 734 is located at the loop between the tracrRNA and the crRNA 730 of the gRNA.

### Base Editing Complexes

According to another embodiment of the present invention, there is a base editing complex. The base editing complex comprises, consists essentially of, or consists of a gRNA-ligand binding complex of the present invention; and a Type V Cas protein, wherein the Cas association region and anti-repeat region of the gRNA-ligand binding complex are associated with the Type V Cas protein. Thus, the gRNA is capable of associating with the Cas protein. After association has occurred, the Cas protein will, based on the identity of targeting sequence, find the target site.

### Type V Cas Protein

In general, a Cas protein includes at least one RNA binding domain. The RNA binding domain interacts with the guide RNA at the Cas association region. The Type V Cas protein that is of use in the present invention is one with which the gRNA-ligand binding complex can associate in the presence of a tracrRNA that contains an anti-repeat region that is of sufficient complementarity to the Cas association region. In some embodiments, the Type V Cas protein is an endonuclese that contains a RuvC domain This RuvC domain can be mutated such that the endonuclease activity is deactivated. In some embodiments, the protein is a nickase that contains an active or deactivated RuvC domain.

Examples of Type V Cas proteins that may be of use in connection with the present invention include, but are not limited to, Cas12b, Cas12e, CasMINI and Cas12f in active or deactivated form.

The Cas protein may be provided in purified or isolated form or can be part of a composition or a complex. Preferably, when in a composition, the protein is first purified to some extent, more preferably to a high level of purity (e.g., about 80%, 90%, 95%, or 99% or higher). Compositions in which the complexes and components of the present invention may be stored and transported may be any type of composition desired, e.g., aqueous compositions suitable for use as, or inclusion in, a composition for RNA-guided targeting that a person of ordinary skill in the art would appreciate would be of use in connection with the present invention.

In some embodiments, the Type V Cas proteins comprise a fusion protein having (a) an active, partially deactivated or deactivated Type V Cas protein, and (b) a uracil DNA glycosylase (UNG) inhibitor peptide (UGI). The UGI peptide can be fused directly to the Type V Cas protein or through a linker peptide comprised of 1 to 100 hundred amino acid residues. In some embodiments, the UGI comprises the wild type UGI sequence from the Bacillus phage PBS2 (https://www.ncbi.nlm.nih.gov/protein/P14739): MTNLSDIIEKETGKQLVIQESILMLPEEVEEVIGNKPESDILVHTAYDESTDENV MLLTSDAPEYKPWALVIQDSNGENKIKML (SEQ ID NO: 22). In some embodiments, the UGI comprises variants of SEQ ID NO: 22 that comprises a fragment of the wild type UGI peptide or a homologous amino acid sequence to SEQ ID NO: 22. In some embodiments, the UGI fragment of homologous sequence comprises at least 60%, at least 65%, at least 70%, at least 75%, at least 80%, at least 85%, at least 90%, at least 95%, at least 97%, at least 99%, or at least 99.5% homology to the wild type UGI peptide sequence (SEQID NO: 22).

In some embodiments, the active or deactivated Type V Cas protein comprises a fusion with two or more UGI peptides or variants. The UGI peptides, or variants of the UGI peptide, can be connected directly to another UGI peptide or Type V Cas protein or via a linker of 1 to 100 amino acid residues to another UGI peptide or Type V Cas protein.

The Cas protein or Cas protein fusion may be provided in purified or isolated form or can be part of a composition or complex.

### Effectors

The base editing complexes of the present invention may contain an effector that is attached to a ligand. The ligand is capable of reversibly or irreversibly associating with the ligand binding moiety. Thus, the ligand binding moiety recruits an effector, e.g. base editing enzyme that is fused to or otherwise associated with the ligand, because the ligand binding moiety is capable of retaining association with the ligand. This design may be particularly advantageous because it provides a modular design in which the nucleic acid sequence targeting function of the gRNA and effector function reside in different molecules. For example, to introduce modifications serially at the same site, one may use different effectors that are associated with the same ligand. Conversely, to introduce the same modifications at different sites, one may use the same ligand binding moiety with different gRNAs while using the same effector-ligand. Thus, this design allows one to multiplex a system without an undesirable burden of fusing effectors to either gRNAs or Cas proteins.

Examples of effectors that may be of use in connection with the present invention are deaminases such as those that have cytidine deamination or adenine deamination activity, as well as transcriptional regulators, repair enzymes, epigenetic modifiers, histone acetylases, deacetylases, methylases (of histones ad nucleotides), and demethylases (of histones and nucleotides). In some embodiments, the effector is selected from the group consisting of AID, CDA, APOBEC1, APOBEC3A, APOBEC3B, APOBEC3C, APOBEC3D, APOBEC3F, ADA, ADAR and tRNA adenosine deaminase. Examples of effectors and the types of genetic change that they case are provided in table 1.

TABLE 1
Examples of effector proteins

| Enzyme type | Genetic change | Effector protein abbreviated |
|---|---|---|
| Cytidine deaminase | C→U/T | AID |
| | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| Adenosine | A→I/G | ADA |
| deaminase | | ADAR1 |
| | | TadA |
| | | TADA |
| | | TAD3 |
| DNA Methyl | C→Met-C | ADAR2 |
| transferase | | ADAR3 |
| | | Dnmt1 |
| Demethylase | Met-C→ C | Dnmt3a |
| Cytidine | 5mC → 5hmC | TET1 |
| demethylase | | |
| Cytidine | 5mC → 5hmC | TET2 |
| demethylase | 5hmC → 5fC/5caC | |
| Glycosylase | 5fc/5caC → C | TDG |

Effector protein full names:
AID: activation induced cytidine deaminase, a.k.a AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
ADA: adenosine deaminase
ADAR1: adenosine deaminase acting on RNA 1
ADAR2: adenosine deaminase acting on RNA 2
ADAR3: adenosine deaminase acting on RNA 3
Dnmt1: DNA (cytosine-5-)-methyltransferase 1
Dnmt3a: DNA (cytosine-5-)-methyltransferase 3 alpha
TadA: tRNA-specific adenosine deaminase
TADA: tRNA(adenine(34)) deaminase, chloroplastic
TAD3: tRNA-specific adenosine deaminase TAD3
TET1: Methylcytosine dioxygenase TET1
TET2: Methylcytosine dioxygenase TET2
TDG: G/T mismatch-specific thymine DNA glycosylase

In some embodiments, the base editing complex comprises two or more effectors. When there are two effectors they may be referred to as: a first effector and a second effector. Each effector may be attached to a different ligand binding moiety through a different ligand. Alternatively, when there are two effectors present, one is attached to a ligand and associated with the gRNA through the ligand binding moiety and another is attached directly to the Cas protein.

### Ligands

As noted above, the effector is bound to a ligand, e.g., by one or more covalent bonds. A non-exhaustive list of examples of ligand binding moiety-ligand pairs that may be used in various embodiments of the present invention is provided in Table 2. Both unmodified and chemically modified versions or the ligand binding moieties and ligands are within the scope of the present invention.

TABLE 2

| Ligand Binding Moieties | Ligands |
|---|---|
| Telomerase Ku binding motif | Ku |
| Telomerase Sm7 binding motif | Sm7 |
| MS2 phage operator stem-loop | MS2 Coat Protein (MCP) |
| PP7 phage operator stem-loop | PP7 coat protein (PCP) |
| Qbeta phage operator stem-loop | Qbeta coat protein [Q65H] |
| SfMu phage Com stem-loop | Com RNA binding protein |
| Non-natural RNA aptamer | Corresponding aptamer ligand |
| Biotin | Streptavidin |
| Oligosaccharide | Lectin |
| Benzylguanine or benzylcytosine | SNAP/CLIP tag |
| 6x-His binding motif | 6x-His tag |
| PDGFbeta chain binding motif | PDGF B-chain |
| GST binding motif | GST protein |
| Tat binding motif | BIV Tat protein |
| Tat binding motif | HIV Tat protein |
| Pumilio binding motif | PUM-HD domain |
| BoxB binding motif | Lambda N22plus |
| Csy4 binding motif | Csy4[H29A] |

Some of the sequences for the above binding pairs are listed below.

1. Telomerase Ku binding motif/Ku heterodimer
a. Ku binding hairpin
(SEQ ID No: 23)
5′-UUCUUGUCGUACUUAUAGAUCGCUACGUUAUUUCAAUUUUGAAAAU
CUGAGUCCUGGGAGUGCGGA-3′
b. Ku heterodimer
(SEQ ID No: 24)
MSGWESYYKTEGDEEAEEEQEENLEASGDYKYSGRDSLIFLVDASKAMF
ESQSEDELTPFDMSIQCIQSVYISKIISSDRDLLAVVFYGTEKDKNSVN
FKNIYVLQELDNPGAKRILELDQFKGQQGQKRFQDMMGHGSDYSLSEVL
WVCANLFSDVQFKMSHKRIMLFTNEDNPHGNDSAKASRARTKAGDLRDT
GIFLDLMHLKKPGGFDISLFYRDIISIAEDEDLRVHFEESSKLEDLLRK
VRAKETRKRALSRLKLKLNKDIVISVGIYNLVQKALKPPPIKLYRETNE
PVKTKTRTFNTSTGGLLLPSDTKRSQIYGSRQIILEKEETEELKRFDDP
GLMLMGFKPLVLLKKHHYLRPSLFVYPEESLVIGSSTLFSALLIKCLEK
EVAALCRYTPRRNIPPYFVALVPQEEELDDQKIQVTPPGFQLVFLPFAD
DKRKMPFTEKIMATPEQVGKMKAIVEKLRFTYRSDSFENPVLQQHFRNL
EALALDLMEPEQAVDLTLPKVEAMNKRLGSLVDEFKELVYPPDYNPEGK
VTKRKHDNEGSGSKRPKVEYSEEELKTHISKGTLGKFTVPMLKEACRAY
GLKSGLKKQELLEALTKHFQD>
(SEQ ID No: 25)
MVRSGNKAAVVLCMDVGFTMSNSIPGIESPFEQAKKVITMFVQRQVFAE
NKDEIALVLFGTDGTDNPLSGGDQYQNITVHRHLMLPDFDLLEDIESKI
QPGSQQADFLDALIVSMDVIQHETIGKKFEKRHIEIFTDLSSRFSKSQL
DIIIHSLKKCDISERHSIHWPCRLTIGSNLSIRIAAYKSILQERVKKTW
TVVDAKTLKKEDIQKETVYCLNDDDETEVLKEDIIQGFRYGSDIVPFSK
VDEEQMKYKSEGKCFSVLGFCKSSQVQRRFFMGNQVLKVFAARDDEAAA
VALSSLIHALDDLDMVAIVRYAYDKRANPQVGVAFPHIKHNYECLVYVQ
LPFMEDLRQYMFSSLKNSKKYAPTEAQLNAVDALIDSMSLAKKDEKTDT
LEDLFPTTKIPNPRFQRLFQCLLHRALHPREPLPPIQQHIWNMLNPPAE
VTTKSQIPLSKIKTLFPLIEAKKKDQVTAQEIFQDNHEDGPTAK
2. Telomerase Sm7 binding motif/Sm7 homoheptamer
a. Sm consensus site (single stranded)
(SEQ ID No: 26)
5′-AAUUUUUGGA-3′
b. Monomeric Sm-like protein (archaea)
(SEQ ID No: 27)
GSVIDVSSQRVNVQRPLDALGNSLNSPVIIKLKGDREFRGVLKSFDLHM
NLVLNDAEELEDGEVTRRLGTVLIRGDNIVYISP
3. MS2 phage operator stem loop/MS2 coat protein
a. MS2 phage operator stem loop
(SEQ ID No: 28)
5′-GCACAUGAGGAUCACCCAUGUGC-3′
b. MS2 coat protein
(SEQ ID No: 29)
MASNFTQFVLVDNGGTGDVTVAPSNFANGIAEWISSNSRSQAYKVTCSV
RQSSAQNRKYTIKVEVPKGAWRSYLNMELTIPIFATNSDCELIVKAMQG
LLKDGNPIPSAIAANSGIY
4. PP7 phage operator stem loop/PP7 coat protein
a. PP7 phage operator stem loop
(SEQ ID No: 30)
5′-AUAAGGAGUUUAUAUGGAAACCCUUA-3′
b. PP7 coat protein (PCP)
(SEQ ID No: 31)
MSKTIVLSVGEATRTLTEIQSTADRQIFEEKVGPLVGRLRLTASLRQNG
AKTAYRVNLKLDQADVVDCSTSVCGELPKVRYTQVWSHDVTIVANSTEA
SRKSLYDLTKSLVATSQVEDLVVNLVPLGR.
5. SfMu Com stem loop/SfMu Com binding protein
a. SfMu Com stem loop
(SEQ ID No: 32)
5′-CUGAAUGCCUGCGAGCAUC-3′
b. SfMu Com binding protein
(SEQ ID No: 33)
MKSIRCKNCNKLLFKADSFDHIEIRCPRCKRHIIMLNACEHPTEKHCGK
REKITHSDETVRY
6. BoxB aptamer/lambda N22plus
a. BoxB aptamer
(SEQ ID No: 34)
5′-GCCCUGAAGAAGGGC-3′
b. Lambda N22plus protein
(SEQ ID No: 35)
MNARTRRRERRAEKQAQWKAAN
7. Csy4 binding stem loop/Csy4[H29A]
a. Csy4 binding motif
(SEQ ID No: 36)
5′-CUGCCGUAUAGGCAGC-3′
b. Csy4[H29A]
(SEQ ID No: 37)
MDHYLDIRLRPDPEFPPAQLMSVLFGKLAQALVAQGGDRIGVSFPDLDE
SRSRLGERLRIHASADDLRALLARPWLEGLRDHLQFGEPAVVPHPTPYR
QVSRVQAKSNPERLRRRLMRRHDLSEEEARKRIPDTVARALDLPFVTLR
SQSTGQHFRLFIRHGPLQVTAEEGGFTCYGLSKGGFVPWF
8. Qbeta binding stem loop [Q65H]
a. Qbeta phage operator stem loop
(SEQ ID No: 96)
5′-ATGCTGTCTAAGACAGCAT-3′
b. Qbeta coat protein [Q65H]
(SEQ ID No: 97)
MAKLETVTLGNIGKDGKQTLVLNPRGVNPTNGVASLSQAGAVPALEKRV
TVSVSQPSRNRKNYKVHVKIQNPTACTANGSCDPSVTRQAYADVTFSFT
QYSTDEERAFVRTELAALLASPLLIDAIDQLNPAY

In each of the aforementioned sequences, one may, for example, use the identical sequence or sequences that have one or more insertions, deletions or substitutions in one or both sequences of a binding pair. By way of a non-limiting example, for either or both members of a binding pair one may use a sequence that is at least 80%, at least 85%, at least 90%, at least 95%, or at least 98% the same as an aforementioned sequence.

### Additional Chemistries

In some embodiments, the base-editing complexes of the present invention are combined with additional chemistry technologies. For example, in some embodiments, a base editing complex further comprises a cysteine/selenocysteine tag. In some embodiments, the base editing complex comprises or is associated with elements for cycloaddition via click chemistry.

### Methods for Base-Editing

In another embodiment, the present invention provides methods for base editing. In these methods, one exposes a base editing complex of the present invention to double-stranded DNA or to a solution that contains dsDNA or to a cell that contains dsDNA or to a subject. The method may occur in vitro or be conducted in vivo or ex vivo and may comprise delivering the base editing complex to a subject as part of a medicament for treatment.

These methods may, for example, be used to modify an immune cell selected from a T cell (including a primary T cell), Natural Killer (NK cell), B cell, or CD34+ hematopoietic stem progenitor cell (HSPC). The immune cell may be an engineered immune cell, such as T-cell comprising a CAR or TCR. The methods herein may thus be applied to engineer further a cell that has already been modified to include a CAR and/or TCR that is useful in therapy. By way of further example, primary immune cells, either naturally occurring within a host animal or patient, or derived from a stem cell or an induced pluripotent stem cell [iPSC] may be genetically modified using the methods and complexes provided herein. Suitable stem cells include, but are not limited to, mammalian stem cells such as human stem cells, including, but not limited to, hematopoietic, neural, embryonic, induced pluripotent stem cells (iPSC), mesenchymal, mesodermal, liver, pancreatic, muscle, and retinal stem cells. Other stems cells include, but are not limited to, mammalian stem cells such as mouse stem cells, e.g., mouse embryonic stem cells.

Provided herein are also methods for genome engineering (e.g., altering or manipulating the expression of one or more genes or one or more gene products) in prokaryotic or eukaryotic cells, in vitro, in vivo, or ex vivo. In particular, the methods provided herein may be useful for targeted base editing disruption in mammalian cells including primary human T cells, natural killer (NK) cells, CD34+ hematopoietic stem and progenitor cells (HSPCs), such as HSPCs isolated from umbilical cord blood or bone marrow and cells differentiated from them.

Also provided herein are genetically engineered cells arising from haematopoietic stem cells, such as T cells, that have been modified according to the methods described herein.

In some cases, the methods are configured to produce genetically engineered T cells arising from HSCs or iPSCs, that are suitable as “universally acceptable” cells for therapeutic application. Haemopoietic stem cells (HSCs) arise from hemangioblasts, which can give rise to HSCs, vascular smooth muscle cells and angioblasts, which differentiate into vascular endothelial cells. HSCs can give rise to common myeloid and common lymphoid progenitors from which arise T cells, Natural Killer (NK) cells, B cells, myeloblasts, erythroblasts and other cells involved in the production of cells of blood, bone marrow, spleen, lymph nodes, and thymus. Such methods can also be applied to natural killer (NK) cells, CD34+ hematopoietic stem and progenitor cells (HSPCs), such as HSPCs isolated from umbilical cord blood or bone marrow and cells differentiated from them.

In another aspect, provided herein are methods for targeting diseases for base editing correction. In some of the methods, the base editing complexes are delivered to a subject for treatment. The target sequence can be any disease-associated polynucleotide or gene. Examples of useful applications of mutation or correction of an endogenous gene sequence according to the present invention include but are not limited to: alterations of disease-associated gene mutations, alterations in sequences encoding splice sites, alterations in regulatory sequences, alterations in sequences to cause a gain-of-function mutation, and/or alterations in sequences to cause a loss-of-function mutation, and targeted alterations of sequences encoding structural characteristics of a protein.

### Delivery of Components into Cells

The base editing complexes or their components may be delivered to target cells and organisms via various methods and various formats (DNA, RNA or protein) or combination of these different formats. The base editing components may be delivered as: (a) DNA polynucleotides that encode the relevant sequence for the protein effectors or the guide RNAs; (b) synthetic RNA encoding the sequence for the protein effectors (messenger RNA) or the guide RNAs; (c) purified protein for the effectors. When delivering as protein format, the Type V Cas protein can be assembled with the guide RNAs to form a ribonucleoprotein complex (RNP) tor delivery into target cells and organisms.

For example, the components or complexes as assembled may be delivered together or separately by electroporation, by nucleofection, by transfection, via nanoparticles, via viral mediated RNA delivery, via non-viral mediated delivery, via extracellular vesicles (for example, exosome and microvesicles), via eukaryotic cell transfer (for example, by recombinant yeast) and other methods that can package molecules such that they can be delivered to a target viable cell without changes to the genomic landscape.

Other methods include, but are not limited to, non-integrative transient transfer of DNA polynucleotides that include the relevant sequence for the protein recruitment so that the molecule can be transcribed into the desired RNA molecule and for amino acid containing components translated into a protein or protein fragment. This includes, without limitation, DNA-only vehicles (for example, plasmids, MiniCircles, MiniVectors, MiniStrings, Protelomerase generated DNA molecules (for example Doggybones), artificial chromosome (for example HAC), and cosmids), via DNA vehicles by nanoparticles, extracellular vesicles (for example, exosome and microvesicles), via eukaryotic cell transfer (for example, by recombinant yeast), transient viral transfer by AAV, non-integrating viral particles (for example, lentivirus and retrovirus based systems), cell penetrating peptides and other technology that can mediate the introduction of DNA into a cell without direct integration into the genomic landscape. Another method for the introduction of the RNA components include the use of integrative gene transfer technology for stable introduction of the machinery for RNA transcription into the genome of the target cells, this can be controls via constitutive or promoter inducible systems to attenuate the RNA expression and this can also be designed so that the system can be removed after the utility has been met (for example, introducing a Cre-Lox recombination system), such technology for stable gene transfer includes, but is not limited to, integrating viral particles (for example, lentivirus, adenovirus and retrovirus based systems), transposase mediate transfer (for example Sleeping Beauty and Piggybac), exploitation of the non-homologous repair pathways introduced by DNA breaks (for example, utilizing CRISPR and TALEN) technology and a surrogate DNA molecule, and other technology that encourages integration of the target DNA into a cell of interest.

The various components of the complexes of the present invention, if not synthesized enzymatically within a cell or solution, may be created chemically or, if naturally occurring, isolated and purified from naturally occurring sources. Methods for chemically and enzymatically synthesizing the various embodiments of the present invention are well known to persons of ordinary skill in the art. Similarly, methods for ligating or introducing covalent bonds between components of the present invention are also well known to persons of ordinary skill in the art.

### Applications

By way of a non-limiting example, the complexes of the present invention may be used to recruit transcriptional activators such as p65 and V64, as well as moieties that introduce epigenetic modifications or affect HDR. The complexes of the present invention can also be used for the following applications; base editing, genome editing, genome screening, generation of therapeutic cells, genome tagging, epigenome editing, karyotype engineering, chromatin imaging, transcriptome and metabolic pathway engineering, genetic circuits engineering, cell signaling sensing, cellular events recording, lineage information reconstruction, gene drive, DNA genotyping, miRNA quantification, in vivo cloning, site-directed mutagenesis, genomic diversification, and proteomic analysis in situ. In some embodiments, a cell or a population of cells are exposed to a base editing complex of the present invention and the cell or cells are introduced to a subject by infusion.

Applications also include research of human diseases such as cancer immunotherapy, antiviral therapy, bacteriophage therapy, cancer diagnosis, pathogen screening, microbiota remodeling, stem-cell reprogramming, immunogenomic engineering, vaccine development, and antibody production.

### EXAMPLES

### Example 1: Transfection of Plasmid Components for Cas12e Base Editing in Mammalian Cells (Prophetic)

### Vector Construction

The coding sequence for the Cas may be synthetically obtained and cloned into a vector under the control of the mouse CMV promoter (mCMV) in a T2A polycistronic cassette with a red fluorescent protein-puromycin fusion. A deactivated version of the Cas and 2xUGI fusion to the deactivated Cas12e variants may also be obtained and cloned in the aforementioned vector. The coding sequence for MS2 coat protein APOBEC fusion (MCP-APOBEC) may be obtained and cloned into an expression vector under control of the mouse CMV promoter. The sequence for gRNAs containing the MS2 ligand binding moiety and unique spacer regions may be cloned into an expression vector under control of the hU6 promoter.

### Transfection:

HEK 293T cells (ATCC, #CRL-11268) may be seeded at 20,000 cells per well in a 96-well plate one day prior to transfection. Cells may be co-transfected using DharmaFECT Duo Transfection Reagent (Horizon Discovery, #T-2010) and 200 ng Cas12e plasmid, or its deactivated variants fused or not with UGI, 50 ng MCP-APOBEC plasmid, and 50 ng gRNA plasmid. The gRNA plasmid may consist of a constant region length of 101 nucleotides, different spacer sequences targeting transcripts within PPIB or EMX1 gene targets, and have the MS2 ligand binding moiety at the 5′ terminus, the 3′ terminus, internally not at either the 5′ or 3′ terminus, or combinations therein.

Cells may be selected in puromycin containing media and harvested 48 hours post-transfection for further processing as described below and the following sequences may be used.

Cas12e gRNA sequences:
(SEQ ID No: 38)
5′-GCGCACATGAGGATCACCCATGTGCGGCGCGUUUAUUCCAUUACUU
UGGAGCCAGUCCCAGCGACUAUGUCGUAUGGACGAAGCGCUUAUUUAUC
GGAGAGAAACCGAUAAGUAAAACGCAUCAAANNNNNNNNNNNNNNNNNN
NNN-3′
(SEQ ID No: 39)
5′-GGCGCGUUUAUUCCAUUACUUUGGAGCCAGUCCCAGCGACUGCGCA
CATGAGGATCACCCATGTGCUGUCGUAUGGACGAAGCGCUUAUUUAUCG
GAGAGAAACCGAUAAGUAAAACGCAUCAAANNNNNNNNNNNNNNNNNNN
NN-3′
(SEQ ID No: 40)
5′-GGCGCGUUUAUUCCAUUACUUUGGAGCCAGUCCCAGCGACUAUGUC
GUAUGGACGAAGCGCUUAUUUAUCGGAGAGCGCACATGAGGATCACCCA
TGTGCAAACCGAUAAGUAAAACGCAUCAAANNNNNNNNNNNNNNNNNNN
NN-3′
(SEQ ID No: 41)
5′-GGCGCGUUUAUUCCAUUACUUUGGAGCCAGUCCCAGCGACUAUGUC
GUAUGGACGAAGCGCUUAUUUAUCGGAGAGAAACCGAUAAGUAAAACGC
AUCAAANNNNNNNNNNNNNNNNNNNNNGCGCACATGAGGATCACCCATG
TGC-3′
(SEQ ID No: 42)
5′-GCGCACATGAGGATCACCCATGTGCGGCGCGUUUAUUCCAUUACUU
UGGAGCCAGUCCCAGCGACUAUGUCGUAUGGACGAAGCGCUUAUUUAUC
GGAGAGCGCACATGAGGATCACCCATGTGCAAACCGAUAAGUAAAACGC
AUCAAANNNNNNNNNNNNNNNNNNNNN-3′
EMX1 target sequences:
(SEQ ID No: 43)
5′-AGAACCGGAGGACAAAGTAC-3′
(SEQ ID No: 44)
5′-TGGCAATGCGCCACCGGTTG-3′
(SEQ ID No: 45)
5′-TCTTCTGCTCGGACTCAGGC-3′
(SEQ ID No: 46)
5′-TCTGCTCGGACTCAGGCCCT-3′
(SEQ ID No: 47)
5′-CCAGCTTCTGCCGTTTGTAC-3′
PPIB target sequences
(SEQ ID No: 48)
5′-AAAAACAGTGGATAATTTTG-3′
(SEQ ID No: 49)
5′-GAAGAGACCAAAGATCACCC-3′
(SEQ ID No: 50)
5′-CCTCCGCCTGTGGATGCTGC-3′
(SEQ ID No: 51)
5′-TCCTGCTGCTGCCGGGACCT-3′
(SEQ ID No: 52)
5′-GCGGCCGATGAGAAGAAGAA-3′

### Example 2: Electroporation of mRNA and Synthetic Guides for Cas12e Base Editing in Mammalian Cells (Prophetic)

mRNA Preparation:

Messenger mRNA may be prepared from DNA vectors carrying the T7 promoter and the coding sequences for Cas12e, dCas12e-UGI and MCP-APOBEC following the standard protocols for mRNA in vitro transcription.

### RNA Synthesis:

The crRNA may be synthesized by Horizon Discovery using either 2′-acetoxy ethyl orthoester (2′-ACE) or 2′-tert-butyldimethylsilyl (2′-TBDMS) protection chemistries. RNA oligos may be 2′-depotected/desalted and purified by either high-performance liquid chromatography (HPLC) or polyacrylamide gel electrophoresis (PAGE). Oligos may be resuspended in 10 mM Tris pH7.5 buffer prior to electroporation.

HEK 293T cells (ATCC, #CRL-11268) may be electroporated using the Invitrogen™ Neon™ Transfection System, 10 μl Kit. A mixture of 50,000 cells, 1 μg of Cas12e or dCas12e-UGI mRNA and MCP-APOBEC mRNA, and 3 μM of synthetic crRNA and tracrRNA may be electroporated at 1150V for 20 ms and for 2 pulses. The chemically synthesized crRNA may consist of a constant region length of 23 nucleotides, different spacer sequences targeting transcripts within PPIB or EMX1 gene targets, and have the MS2 ligand binding moiety at the 5′ terminus, the 3′ terminus, internally not at either the 5′ or 3′ terminus, or combinations therein. Each sequence may contain chemical modifications at one or more bases and within one or more linkages. Cells may be plated in a 96-well plate with full serum media and harvested after 72 hours for further processing. The sequences below may be used.

Cas12e crRNA sequences (N, target sequence):
(SEQ ID No: 53)
5′-GCGCACATGAGGATCACCCATGTGCCCGAUAAGUAAAACGCAUCAA
AGNNNNNNNNNNNNNNNNNNNNN-3′
(SEQ ID No: 54)
5′-CCGAUAAGUAAAACGCAUCAAAGNNNNNNNNNNNNNNNNNNNNNGC
GCACATGAGGATCACCCATGTGC-3′
Cas12e tracrRNA sequences (N, target sequence):
(SEQ ID No: 55)
5′-GCGCACATGAGGATCACCCATGTGCGGCGCGUUUAUUCCAUUACUU
UGGAGCCAGUCCCAGCGACUAUGUCGUAUGGACGAAGCGCUUAUUUAUC
GGAGA-3′
(SEQ ID No: 56)
5′-GGCGCGUUUAUUCCAUUACUUUGGAGCCAGUCCCAGCGACUGCGCA
CATGAGGATCACCCATGTGCUGUCGUAUGGACGAAGCGCUUAUUUAUCG
GAGA-3′
(SEQ ID NO: 57)
5′-GGCGCGUUUAUUCCAUUACUUUGGAGCCAGUCCCAGCGACUAUGUC
GUAUGGACGAAGCGCUUAUUUAUCGGAGAGCGCACATGAGGATCACCCA
TGTGC-3′

For both examples 1 and 2:

### Cell Processing

Cells may be lysed in 100 μL of a buffer containing proteinase K (Thermo Scientific, #FERE00492), RNase A (Thermo Scientific, #FEREN0531), and Phusion GC buffer (Thermo Scientific, #F-518L) for 30 min at 56° C., followed by a 5 min heat inactivation at 95° C. This cell lysate may be used to generate PCR amplicons spanning the region containing the base editing site(s). Unpurified PCR amplicons between 500-1000 bp in length may be sequenced by Sanger sequencing.

### Editing Analysis

Base editing efficiencies may be calculated using the Chimera analysis tool, an adaptation of the open source tool BEAT ((Xu et al. 2019. BEAT: A Python Program to Quantify Base Editing from Sanger Sequencing. The CRISPR Journal 2, 223-229). Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 20 bp input guide sequence.

### Examples 3-6

The following materials and methods were used in examples 3 — 6.

gRNA Sequences

For examples 3-5 (using Cas12b base editor), gRNA sequences encoded by the sequences listed in table 3 were used. All gRNA designs were based on the A. acidoterrestris Cas12b gRNA consisting of a 91 nt constant gRNA sequence, a target specific 20 nt spacer sequence, and a 7 nt poly-T U6 termination signal. All modifications were made to the constant region of the gRNA and consist of the inclusion of the RNA aptamer hairpins. A single copy of the MS2 hairpin sequence (C5 variant) were incorporated into either the 5′, 3′, stem-loop or internal polyU (internal stretch of UUUUU within the gRNA) of the gRNA. The relevant gRNA sequences were cloned into a separate expression vector under control of the hU6 promoter.

In table 3, N denotes the 20 nt target specific spacer sequence. The constant gRNA sequence as previously described is highlighted in bold. MS2 (C5 variant) is displayed in italics whilst the extensions to the aptamer are shown in italics and underlined. The mutation introduced in the internal polyU stretch is in bold and underlined.

TABLE 3
gRNA sequences for use with Cas12b

| gRNA name | sgRNA sequence [5′ −> 3′] | SEQ ID NO: |
|---|---|---|
| A. acidoterrestris | GTCTAGAGGACAGAATTTTTCAACGGGTGTGCCAATGGCCACTT | 58 |
| gRNA | TCCAGGTGGCAAAGCCCGTTGAGCTTCTCAAATCTGAGAAGTGG | |
| (MS2-less) | CACNNNNNNNNNNNNNNNNNNNNTTTTTTT | |
| 5′MS2 | GGCACATGAGGATCACCCATGTGCGTCTAGAGGACAGAATTTTT | 59 |
| | CAACGGGTGTGCCAATGGCCACTTTCCAGGTGGCAAAGCCCGTT | |
| | GAGCTTCTCAAATCTGAGAAGTGGCACNNNNNNNNNNNNNNNNN | |
| | NNNTTTTTTT | |
| 5′MS2 U −> G | GGCACATGAGGATCACCCATGTGCGTCTAGAGGACAGAATTGTT | 60 |
| | CAACGGGTGTGCCAATGGCCACTTTCCAGGTGGCAAAGCCCGTT | |
| | GAGCTTCTCAAATCTGAGAAGTGGCACNNNNNNNNNNNNNNN | |
| | NNNNNTTTTTTT | |
| 3′MS2 | GGTCTAGAGGACAGAATTTTTCAACGGGTGTGCCAATGGCCACT | 61 |
| | TTCCAGGTGGCAAAGCCCGTTGAGCTTCTCAAATCTGAGAAGTG | |
| | GCACGCACATGAGGATCACCCATGTGCNNNNNNNNNNNNNNNN | |
| | NNNNTTTTTTT | |
| 3′MS2 U −> G | GGTCTAGAGGACAGAATTGTTCAACGGGTGTGCCAATGGCCACT | 62 |
| | TTCCAGGTGGCAAAGCCCGTTGAGCTTCTCAAATCTGAGAAGTG | |
| | GCACGCACATGAGGATCACCCATGTGCNNNNNNNNNNNNNNNN | |
| | NNNNTTTTTTT | |
| Loop MS2 | GGTCTAGAGGACAGAATTTTTCAACGGGTGTGCCAATGGACATG | 63 |
| | AGGATCACCCATGTCCAGGTGGCAAAGCCCGTTGAGCTTCTCAA | |
| | ATCTGAGAAGTGGCACNNNNNNNNNNNNNNNNNNNNTTTTTTT | |
| Loop MS2 U −> G | GGTCTAGAGGACAGAATTGTTCAACGGGTGTGCCAATGGACATG | 64 |
| | AGGATCACCCATGTCCAGGTGGCAAAGCCCGTTGAGCTTCTCAA | |
| | ATCTGAGAAGTGGCACNNNNNNNNNNNNNNNNNNNNTTTTTTT | |
| polyU MS2 | GGTCTAGAGGACAGAATTGCACATGAGGATCACCCATGTGCTTT | 65 |
| | CAACGGGTGTGCCAATGGCCACTTTCCAGGTGGCAAAGCCCGTT | |
| | GAGCTTCTCAAATCTGAGAAGTGGCACNNNNNNNNNNNNNNNNN | |
| | NNNTTTTTTT | |

For example 6 (using CasMINI base editor), gRNA sequences encoded by the sequences listed in table 4 were used. All gRNA designs were based on the Acidibacillus sulfuroxidans Cas12f gRNA consisting of a 162 nt constant gRNA sequence, a target specific 23 nt spacer sequence, and a 7 nt poly-T U6 termination signal. All modifications were made to the constant component of the gRNA and consist of the inclusion of the RNA aptamer hairpins and truncations of stem-loops. A single copy of the MS2 hairpin sequence (C5 variant) was incorporated into either the 5′, 5′ and a truncation of the stem loop 1, stem loop 1 extension, replacement of stem loop 2 and truncation of stem loop 1, or replacement of the repeat:antirepeat. The relevant gRNA sequences were cloned into a separate expression vector under control of the hU6 promoter.

In table 4, N denotes the 23 nt target specific spacer sequence. The constant gRNA sequence is highlighted in bold. The MS2 (C5 variant) is displayed in italics whilst the extensions to the aptamer are shown in italics and underlined. Design 1 corresponds to a 5′MS2, design 2 corresponds to 5′MS2 and a stem loop 1 truncation, design 3 corresponds to an MS2 located as an extension of stem loop 1, design 4 corresponds to a MS2 located as a replacement of stem loop 2 and a truncation of stem loop 1, design 5 corresponds to a MS2 located as a replacement of the repeat:antirepeat region.

TABLE 4
gRNA sequences for use with CasMINI

| gRNA name | gRNA sequence [5′ −> 3′] | SEQ ID NO: |
|---|---|---|
| A. sulforoxidans | ATGGGCTTCACTGATAAAGTGGAGAACCGCTTCACCAA | 66 |
| gRNA | AAGCTGTCCCTTAGGGGATTAGAACTTGAGTGAAGGTG | |
| (MS2-less) | GGCTGCTTGCATCAGCCTAATGTCGAGAAGTGCTTTCT | |
| | TCGGAAAGTAACCCTCGAAACAAATTCATTTGAATGAA | |
| | GGAATGCAACNNNNNNNNNNNNNNNNNNNNNNNTTTTT | |
| | TT | |
| Design 1 | GCACATGAGGATCACCCATGTGCATGGGCTTCACTGAT | 67 |
| | AAAGTGGAGAACCGCTTCACCAAAAGCTGTCCCTTAGG | |
| | GGATTAGAACTTGAGTGAAGGTGGGCTGCTTGCATCAG | |
| | CCTAATGTCGAGAAGTGCTTTCTTCGGAAAGTAACCCT | |
| | CGAAACAAATTCATTTGAATGAAGGAATGCAACNNNNN | |
| | NNNNNNNNNNNNNNNNNNTTTTTTT | |
| Design 2 | GCACATGAGGATCACCCATGTGCCGCTTCACCAAAAGC | 68 |
| | TGTCCCTTAGGGGATTAGAACTTGAGTGAAGGTGGGCT | |
| | GCTTGCATCAGCCTAATGTCGAGAAGTGCTTTCTTCGG | |
| | AAAGTAACCCTCGAAACAAATTCATTTGAATGAAGGAA | |
| | TGCAACNNNNNNNNNNNNNNNNNNNNNNNTTTTTTT | |
| Design 3 | GGGGCACATGAGGATCACCCATGTGCAACCGCTTCACC | 69 |
| | AAAAGCTGTCCCTTAGGGGATTAGAACTTGAGTGAAGG | |
| | TGGGCTGCTTGCATCAGCCTAATGTCGAGAAGTGCTTT | |
| | CTTCGGAAAGTAACCCTCGAAACAAATTCATTTGAATG | |
| | AAGGAATGCAACNNNNNNNNNNNNNNNNNNNNNNNTTT | |
| | TTTT | |
| Design 4 | ACCGCTTCACGCACATGAGGATCACCCATGTGCGTGAA | 70 |
| | GGTGGGCTGCTTGCATCAGCCTAATGTCGAGAAGTGCT | |
| | TTCTTCGGAAAGTAACCCTCGAAACAAATTCATTTGAA | |
| | TGAAGGAATGCAACNNNNNNNNNNNNNNNNNNNNNNNT | |
| | TTTTTT | |
| Design 5 | GGGCTTCACTGATAAAGTGGAGAACCGCTTCACCAAAA | 71 |
| | GCTGTCCCTTAGGGGATTAGAACTTGAGTGAAGGTGGG | |
| | CTGCTTGCATCAGCCTAATGTCGAGAAGTGCTTTCTTC | |
| | GGAAAGTAACCCTCGAAACAAAGCACATGAGGATCACC | |
| | CATGTGCGGAATGCAACNNNNNNNNNNNNNNNNNNNNN | |
| | NNTTTTTTT | |

### Plasmid Design

Aside from the gRNA, all components of the systems used were encoded on two vectors and expressed from a CMV promoter. The first vector encoded an enhanced human Apobec3A-MCP or Anolis carolinensis Apobec1a-MCP fusion protein (Deaminase vector). The second vector encoded a dCas12b (D569A, E847A, D976A) or dCasMINI (D325A; D509A) fused to two copies of UGI through its C-terminus (Cas vector). dCasMINI is a previously described version of dUn1Cas12f1. The dCas12b-UGI-UGI and dCasMINI-UGI-UGI fusion proteins were flanked by 2 copies of the SV40 NLS at the N terminus of the Cas sequence and the C terminus of UGI sequence. Additionally, the vector encoded the expression of turboRFP to allow the monitoring of transfection efficiency.

The relevant gRNA sequences were cloned into a separate expression vector under control of the hU6 promoter (gRNA expression vector).

One plasmid, which appears below as SEQ ID NO: 72 and was used in examples 3, 4, and 5, encodes a dead Cas12b fused to two UGIs: dCas12b-UGI-UGI. Below the following fonts are used:
normal humanized Cas12b
italic: SV40 NLS
underlined: linker1
italic and underlined: linker2
Double underlined: UGI
bold and underlined: mutations c.1706 A>C; c.2540 A>C; c.2927 A>C

SEQ ID NO: 72:
ATGGCCCCAAAGAAGAAGCGGAAAGTCGCCGTGAAAAGCATTAAAGTG
AAACTGAGGCTGGACGATATGCCCGAAATCAGAGCCGGCCTCTGGAAG
CTGCACAAGGAGGTCAACGCCGGCGTTCGTTATTACACAGAGTGGCTG
TCTTTACTCAGACAAGAAAATTTATATAGAAGGAGCCCCAATGGCGAT
GGCGAGCAAGAGTGCGACAAAACCGCCGAAGAATGCAAGGCCGAACTG
CTCGAAAGACTGAGAGCCAGACAAGTTGAGAACGGACACAGAGGACCC
GCCGGCTCCGATGATGAACTGCTGCAGCTCGCTAGGCAACTGTACGAG
CTGCTCGTCCCCCAAGCTATCGGAGCTAAAGGAGACGCTCAGCAGATC
GCTAGGAAGTTTCTGAGCCCCCTCGCCGATAAAGACGCTGTGGGCGGT
TTAGGAATCGCCAAGGCTGGAAATAAACCTCGTTGGGTGAGGATGAGG
GAAGCTGGCGAGCCCGGCTGGGAGGAGGAAAAGGAGAAAGCCGAAACT
CGTAAATCCGCCGACAGAACAGCTGACGTGCTGAGGGCCCTCGCCGAC
TTCGGTTTAAAACCCCTCATGAGAGTCTATACCGACTCCGAGATGTCC
AGCGTCGAGTGGAAACCCTTACGTAAGGGCCAAGCTGTGAGAACATGG
GATCGTGATATGTTCCAGCAAGCTATCGAAAGGATGATGAGCTGGGAG
TCTTGGAATCAGAGAGTGGGCCAAGAATACGCCAAACTCGTGGAACAG
AAGAATCGTTTCGAGCAGAAAAACTTTGTCGGACAAGAACATTTAGTG
CATCTCGTGAACCAACTGCAGCAAGATATGAAGGAGGCCTCCCCCGGT
TTAGAGAGCAAAGAGCAAACCGCTCACTATGTCACCGGTCGTGCCCTC
AGAGGCTCCGACAAAGTGTTCGAAAAGTGGGGAAAGCTGGCCCCCGAC
GCTCCTTTCGATTTATACGACGCCGAGATCAAGAACGTGCAGAGGAGA
AATACAAGGAGGTTTGGCTCCCACGATTTATTCGCTAAACTCGCCGAG
CCCGAATACCAAGCTTTATGGAGAGAGGATGCCAGCTTTCTGACTCGT
TACGCCGTGTACAATTCCATTCTGAGAAAACTGAACCACGCCAAAATG
TTTGCCACATTCACTTTACCCGATGCCACCGCCCACCCTATCTGGACA
AGATTCGACAAGCTCGGCGGAAACCTCCACCAGTATACCTTTTTATTT
AACGAATTCGGAGAGAGGAGGCATGCCATTCGTTTTCACAAGTTATTA
AAGGTCGAGAATGGAGTCGCCAGAGAGGTGGACGACGTCACCGTGCCT
ATCAGCATGAGCGAACAGCTGGACAATTTACTGCCTCGTGACCCCAAC
GAACCTATCGCCCTCTACTTCAGAGACTACGGAGCTGAGCAGCATTTC
ACCGGCGAGTTCGGAGGCGCTAAGATCCAGTGTAGAAGGGATCAACTG
GCCCATATGCATAGGAGAAGGGGCGCCAGAGATGTCTATCTGAACGTG
AGCGTTCGTGTCCAAAGCCAAAGCGAGGCCAGAGGAGAAAGGAGACCC
CCCTACGCCGCCGTCTTTAGACTGGTCGGCGACAATCATCGTGCTTTC
GTGCACTTTGATAAGCTGTCCGACTATTTAGCCGAACACCCCGACGAT
GGAAAGCTGGGCAGCGAGGGATTATTAAGCGGCCTCAGAGTCATGAGC
GTGGCTCTGGGCCTCAGAACCAGCGCCTCCATCTCCGTCTTTAGGGTG
GCCAGAAAAGACGAGCTGAAGCCCAACAGCAAGGGAAGGGTGCCCTTT
TTCTTCCCTATCAAGGGCAATGACAATTTAGTGGCCGTGCACGAAAGG
TCCCAGTTATTAAAGCTGCCCGGCGAGACCGAAAGCAAAGATTTAAGG
GCTATCAGAGAGGAGAGACAGAGAACTTTAAGACAGCTGAGAACCCAG
CTGGCTTATCTGAGATTATTAGTCAGATGTGGCAGCGAGGACGTCGGT
CGTAGAGAGAGGAGCTGGGCCAAGCTGATTGAACAACCCGTTGATGCC
GCTAATCACATGACCCCCGATTGGAGGGAAGCTTTCGAGAACGAGCTG
CAGAAACTGAAGTCTTTACACGGCATTTGCAGCGACAAGGAGTGGATG
GACGCCGTGTACGAGTCCGTGAGAAGGGTGTGGAGGCACATGGGAAAG
CAAGTTAGGGATTGGAGGAAAGATGTGAGGTCCGGCGAAAGACCCAAG
ATCAGAGGCTACGCCAAGGACGTGGTCGGAGGAAACTCCATCGAGCAG
ATCGAGTACCTCGAGAGACAATACAAGTTTTTAAAGTCTTGGTCCTTC
TTCGGCAAGGTCAGCGGCCAAGTCATTCGTGCTGAAAAGGGATCTCGT
TTCGCCATCACACTGAGAGAGCACATTGACCACGCCAAAGAGGATCGT
CTGAAAAAACTCGCCGATCGTATCATTATGGAGGCCCTCGGCTATGTC
TATGCTCTGGACGAGAGAGGCAAGGGAAAATGGGTCGCCAAGTATCCC
CCTTGTCAACTGATTTTATTAGCGGAGCTGTCCGAGTACCAATTTAAC
AACGATAGGCCTCCCTCCGAGAATAACCAGCTCATGCAGTGGAGCCAT
CGTGGCGTGTTTCAAGAACTGATCAATCAAGCTCAAGTTCACGATTTA
CTCGTGGGCACCATGTACGCCGCTTTTAGCTCCAGATTCGACGCTAGG
ACCGGCGCCCCCGGTATTAGGTGTAGAAGAGTGCCCGCTCGTTGCACC
CAAGAACATAACCCCGAACCCTTTCCTTGGTGGCTGAATAAGTTCGTC
GTCGAGCACACCCTCGACGCTTGCCCTTTACGTGCCGACGACCTCATC
CCTACTGGTGAAGGCGAGATCTTTGTGAGCCCTTTCAGCGCTGAGGAA
GGCGATTTTCACCAGATCCACGCCGCTCTGAACGCCGCCCAGAATCTG
CAACAGAGGCTCTGGAGCGACTTCGATATTTCCCAGATTCGTCTGAGG
TGCGATTGGGGAGAGGTGGATGGAGAACTCGTCCTCATCCCCAGACTG
ACTGGTAAGAGGACCGCTGACAGCTATTCCAATAAGGTCTTCTATACC
AATACTGGTGTCACCTACTACGAGAGAGAGAGGGGCAAGAAGAGAAGG
AAAGTCTTCGCCCAAGAGAAGCTGTCCGAGGAGGAGGCCGAATTATTA
GTCGAGGCTGATGAGGCTCGTGAAAAGTCCGTGGTTTTAATGAGGGAC
CCCTCCGGCATCATCAACAGAGGCAATTGGACTCGTCAGAAGGAATTC
TGGAGCATGGTGAATCAGAGGATCGAGGGCTATCTGGTGAAGCAGATT
AGATCTCGTGTGCCTCTGCAAGATAGCGCTTGTGAGAATACCGGAGAT
ATTAGCGGCGGGAGCGGCGGGAGCGGGGGGAGCACTAATCTGAGCGAC
ATCATTGAGAAGGAGACTGGGAAACAGCTGGTCATTCAGGAGTCCATC
CTGATGCTGCCTGAGGAGGTGGAGGAAGTGATCGGCAACAAGCCAGAG
TCTGACATCCTGGTGCACACCGCCTACGACGAGTCCACAGATGAGAAT
GTGATGCTGCTGACCTCTGACGCCCCCGAGTATAAGCCTTGGGCCCTG
GTCATCCAGGATTCTAACGGCGAGAATAAGATCAAGATGCTGAGCGGA
GGATCCGGAGGATCTGGAGGCAGCACCAACCTGTCTGACATCATCGAG
AAGGAGACAGGCAAGCAGCTGGTCATCCAGGAGAGCATCCTAATGCTT
CCCGAAGAAGTCGAAGAAGTGATCGGAAACAAGCCTGAGAGCGATATC
CTGGTCCATACTGCGTATGATGAAAGTACCGACGAAAACGTAATGCTA
CTCACATCCGACGCCCCAGAGTATAAGCCCTGGGCTCTAGTTATACAA
GACTCCAACGGAGAGAACAAAATCAAAATGCTGTCTGGCGGCTCAAAA
AGAACCGCCGACGGCAGCGAATTCGAGCCCAAGAAGAAGAGGAAAGTC
TAA

The corresponding amino acid sequence for SEQ ID NO: 72 appears below as SEQ ID NO: 73, and in that sequence the following fonts are used:
normal: Cas12b
italic: SV40 NLS
underlined: linker1
italic and underlined: linker2
Double underlined: UGI
bold and underlined: mutations D569A, E847A, D976A

SEQ ID NO: 73 is:
MAPKKKRKVAVKSIKVKLRLDDMPEIRAGLWKLHKEVNAGVRYYTEWLS
LLRQENLYRRSPNGDGEQECDKTAEECKAELLERLRARQVENGHRGPAG
SDDELLQLARQLYELLVPQAIGAKGDAQQIARKFLSPLADKDAVGGLGI
AKAGNKPRWVRMREAGEPGWEEEKEKAETRKSADRTADVLRALADFGLK
PLMRVYTDSEMSSVEWKPLRKGQAVRTWDRDMFQQAIERMMSWESWNQR
VGQEYAKLVEQKNRFEQKNFVGQEHLVHLVNQLQQDMKEASPGLESKEQ
TAHYVTGRALRGSDKVFEKWGKLAPDAPFDLYDAEIKNVQRRNTRRFGS
HDLFAKLAEPEYQALWREDASFLTRYAVYNSILRKLNHAKMFATFTLPD
ATAHPIWTRFDKLGGNLHQYTFLFNEFGERRHAIRFHKLLKVENGVARE
VDDVTVPISMSEQLDNLLPRDPNEPIALYFRDYGAEQHFTGEFGGAKIQ
CRRDQLAHMHRRRGARDVYLNVSVRVQSQSEARGERRPPYAAVFRLVGD
NHRAFVHFDKLSDYLAEHPDDGKLGSEGLLSGLRVMSVALGLRTSASIS
VFRVARKDELKPNSKGRVPFFFPIKGNDNLVAVHERSQLLKLPGETESK
DLRAIREERQRTLRQLRTQLAYLRLLVRCGSEDVGRRERSWAKLIEQPV
DAANHMTPDWREAFENELQKLKSLHGICSDKEWMDAVYESVRRVWRHMG
KQVRDWRKDVRSGERPKIRGYAKDVVGGNSIEQIEYLERQYKFLKSWSF
FGKVSGQVIRAEKGSRFAITLREHIDHAKEDRLKKLADRIIMEALGYVY
ALDERGKGKWVAKYPPCQLILLAELSEYQFNNDRPPSENNQLMQWSHRG
VFQELINQAQVHDLLVGTMYAAFSSRFDARTGAPGIRCRRVPARCTQEH
NPEPFPWWLNKFVVEHTLDACPLRADDLIPTGEGEIFVSPFSAEEGDFH
QIHAALNAAQNLQQRLWSDFDISQIRLRCDWGEVDGELVLIPRLTGKRT
ADSYSNKVFYTNTGVTYYERERGKKRRKVFAQEKLSEEEAELLVEADEA
REKSVVLMRDPSGIINRGNWTRQKEFWSMVNQRIEGYLVKQIRSRVPLQ
DSACENTGDISGGSGGSGGSTNLSDIIEKETGKQLVIQESILMLPEEVE
EVIGNKPESDILVHTAYDESTDENVMLLTSDAPEYKPWALVIQDSNGEN
KIKMLSGGSGGSGGSTNLSDIIEKETGKQLVIQESILMLPEEVEEVIGN
KPESDILVHTAYDESTDENVMLLTSDAPEYKPWALVIQDSNGENKIKML
SGGSKRTADGSEFEPKKKRKV

A second plasmid, which appears below as SEQ ID NO: 74 and was used in example 6, encodes a dead CasMINI fused to two UGIs: dCasMINI-UGI-UGI. Below the following fonts are used:
normal: humanized CasMINI
italic: SV40 NLS
underlined: linker1
italic and underlined: linker2
Double underlined: UGI
bold and underlined: mutations c.974 A>C; c.975 T>A; c.1526 A>C; c.1527 T>G

SEQ ID NO: 74:
ATGGCCCCCAAGAAAAAACGCAAGGTGGCCAAAAACACCATTACCAAAA
ACACTGAAACTGCGTATTGTGCGTCCGTATAATAGCGCAGAAGTGGAAA
AAATTGTTGCCGACGAAAAAAACAACCGCGAAAAAATCGCACTGGAAAA
GAACAAAGACAAAGTGAAAGAAGCCTGCAGCAAACATCTGAAAGTTGCA
GCATATTGTACCACACAGGTTGAACGTAATGCATGCCTGTTTTGTAAAG
CACGTAAACTGGATGACAAATTCTACCAAAAACTGCGTGGTCAGTTTCC
GGATGCAGTTTTTTGGCAAGAAATCAGCGAAATTTTTCGCCAGCTGCAG
AAACAGGCAGCAGAAATCTATAATCAGAGCCTGATCGAACTGTACTACG
AGATTTTTATCAAAGGCAAAGGTATTGCAAATGCCAGCAGCGTTGAACA
TTATCTGAGTAGAGTTTGTTATAGACGTGCAGCAGAACTGTTTAAAAAC
GCAGCAATTGCAAGCGGTCTGCGTAGCAAAATCAAAAGCAATTTTCGTC
TGAAAGAACTGAAAAACATGAAAAGTGGTCTGCCGACCACCAAAAGCGA
TAATTTTCCGATTCCGCTGGTTAAACAGAAAGGTGGTCAGTATACCGGT
TTTGAAATTAGCAATCATAATAGCGACTTCATCATCAAGATTCCGTTTG
GTCGTTGGCAGGTCAAAAAAGAGATTGATAAATATCGTCCGTGGGAGAA
ATTTGACTTTGAACAGGTTCAGAAAAGCCCGAAACCGATTAGCCTGCTG
CTGAGCACCCAGCGTCGTAAACGTAATAAAGGTTGGAGCAAAGATGAAG
GCACCGAAGCCGAAATCAAAAAAGTTATGAATGGCGATTATCAGACCAG
CTACATTGAAGTTAAACGTGGCAGCAAAATCTGTGAAAAAAGCGCATGG
ATGCTGAATCTGAGCATTGATGTTCCGAAAATTGATAAAGGTGTGGATC
CGAGCATTATTGGTGGTATTGCAGTTGGTGTTAGATCACCGCTGGTTTG
CGCAATTAACAATGCATTTAGCCGTTATAGCATCAGCGATAACGACCTG
TTTCACTTCAACAAGAAAATGTTTGCACGTCGTCGTATCCTGCTGAAAA
AAAACCGTCATAAACGTGCAGGTCATGGTGCAAAAAACAAACTGAAACC
GATCACCATTCTGACCGAAAAAAGTGAACGTTTTCGCAAAAAGCTGATT
GAACGTTGGGCATGTGAAATCGCGGATTTCTTCATTAAAAACAAAGTTG
GCACCGTGCAGATGGAAAATCTGGAAAGCATGAAACGTAAAGAGGACAG
CTATTTTAACATTCGCCTGCGTGGCTTTTGGCCGTATGCAGAAATGCAG
AACAAAATCGAATTCAAACTGAAGCAGTATGGCATCGAAATTCGTAAAG
TTGCACCGAATAATACCAGCAAAACCTGTAGCAAATGTGGCCATCTGAA
CAACTATTTCAACTTCGAGTACCGCAAGAAAAACAAATTCCCGCACTTT
AAATGCGAAAAATGCAACTTCAAAGAAAACGCCGCGTATAATGCAGCCC
TGAATATTTCAAACCCGAAACTGAAAAGCACCAAAGAGAGACCGAGCGG
CGGGAGCGGCGGGAGCGGGGGGAGCACTAATCTGAGCGACATCATTGAG
AAGGAGACTGGGAAACAGCTGGTCATTCAGGAGTCCATCCTGATGCTGC
CTGAGGAGGTGGAGGAAGTGATCGGCAACAAGCCAGAGTCTGACATCCT
GGTGCACACCGCCTACGACGAGTCCACAGATGAGAATGTGATGCTGCTG
ACCTCTGACGCCCCCGAGTATAAGCCTTGGGCCCTGGTCATCCAGGATT
CTAACGGCGAGAATAAGATCAAGATGCTGAGCGGAGGATCCGGAGGATC
TGGAGGCAGCACCAACCTGTCTGACATCATCGAGAAGGAGACAGGCAAG
CAGCTGGTCATCCAGGAGAGCATCCTGATGCTGCCCGAAGAAGTCGAAG
AAGTGATCGGAAACAAGCCTGAGAGCGATATCCTGGTCCATACCGCCTA
CGACGAGAGTACCGACGAAAATGTGATGCTGCTGACATCCGACGCCCCA
GAGTATAAGCCCTGGGCTCTGGTCATCCAGGATTCCAACGGAGAGAACA
AAATCAAAATGCTGTCTGGCGGCTCAAAAAGAACCGCCGACGGCAGCGA
ATTCGAGCCCAAGAAGAAGAGGAAAGTCTAA

The corresponding amino acid sequence for SEQ ID NO: 74 appears below as SEQ ID NO: 75, and in that sequence the following fonts are used:
normal: CasMINI
italic: SV40 NLS
underlined: linker1
italic and underlined: linker2
Double underlined: UGI
bold and underlined: mutations D569A, E847A, D976A

SEQ ID NO: 75 is:
MAPKKKRKVAKNTITKTLKLRIVRPYNSAEVEKIVADEKNNREKIALEK
NKDKVKEACSKHLKVAAYCTTQVERNACLFCKARKLDDKFYQKLRGQFP
DAVFWQEISEIFRQLQKQAAEIYNQSLIELYYEIFIKGKGIANASSVEH
YLSRVCYRRAAELFKNAAIASGLRSKIKSNFRLKELKNMKSGLPTTKSD
NFPIPLVKQKGGQYTGFEISNHNSDFIIKIPFGRWQVKKEIDKYRPWEK
FDFEQVQKSPKPISLLLSTQRRKRNKGWSKDEGTEAEIKKVMNGDYQTS
YIEVKRGSKICEKSAWMLNLSIDVPKIDKGVDPSIIGGIAVGVRSPLVC
AINNAFSRYSISDNDLFHFNKKMFARRRILLKKNRHKRAGHGAKNKLKP
ITILTEKSERFRKKLIERWACEIADFFIKNKVGTVQMENLESMKRKEDS
YFNIRLRGFWPYAEMQNKIEFKLKQYGIEIRKVAPNNTSKTCSKCGHLN
NYFNFEYRKKNKFPHFKCEKCNFKENAAYNAALNISNPKLKSTKERPSG
GSGGSGGSTNLSDIIEKETGKQLVIQESILMLPEEVEEVIGNKPESDIL
VHTAYDESTDENVMLLTSDAPEYKPWALVIQDSNGENKIKMLSGGSGGS
GGSTNLSDIIEKETGKQLVIQESILMLPEEVEEVIGNKPESDILVHTAY
DESTDENVMLLTSDAPEYKPWALVIQDSNGENKIKMLSGGSKRTADGSE
FEPKKKRKV

A third plasmid, which appears below as SEQ ID NO: 76, encodes for a deaminase fused to MCP, Enhanced human Apobec3A-MCP. Below the following fonts are used:
normal: human Apobec3A
italic: SV40 NLS
underlined: L25-liner
Double underlined: MCP
bold and underlined: mutations c.307 T>G; c.308 G>C; c.391 T>G

SEQ ID NO: 76:
5′-ATGGCCCCCAAGAAGAAGCGGAAAGTGGAAGCCAGCCCAGCATCCG
GGCCCAGACACTTGATGGATCCACACATATTCACTTCCAACTTTAACAA
TGGCATTGGAAGGCATAAGACCTACCTGTGCTACGAAGTGGAGCGCCTG
GACAATGGCACCTCGGTCAAGATGGACCAGCACAGGGGCTTTCTACACA
ACCAGGCTAAGAATCTTCTCTGTGGCTTTTACGGCCGCCATGCGGAGCT
GCGCTTCTTGGACCTGGTTCCTTCTTTGCAGTTGGACCCGGCCCAAATC
TACAGGGTCACTTGGTTCATCTCCTGGAGCCCCTGCTTCTCCGCGGGCT
GTGCCGGGGAAGTGCGTGCGTTCCTTCAGGAGAACACACACGTGAGACT
GCGTATCTTCGCTGCCCGCATCTATGATGACGACCCCCTATATAAGGAG
GCACTGCAAATGCTGCGGGATGCTGGGGCCCAAGTCTCCATCATGACCT
ACGATGAATTTAAGCACTGCTGGGACACCTTTGTGGACCACCAGGGATG
TCCCTTCCAGCCCTGGGATGGACTAGATGAGCACAGCCAAGCCCTGAGT
GGGAGGCTGCGGGCCATTCTCCAGAATCAGGGAAACGAGCTGAAGACAC
CCCTGGGCGACACCACACACACCTCTCCACCTTGCCCAGCACCAGAGCT
GCTGGGAGGCCCTATGGCCAGCAACTTCACACAGTTTGTGCTGGTGGAT
AATGGAGGAACCGGCGACGTGACAGTGGCACCATCTAACTTTGCCAATG
GCATCGCCGAGTGGATCAGCTCCAACTCTCGGAGCCAGGCCTATAAGGT
GACCTGTAGCGTGCGGCAGTCTAGCGCCCAGAATAGAAAGTATACAATC
AAGGTGGAGGTGCCTAAGGGCGCCTGGAGATCCTACCTGAACATGGAGC
TGACCATCCCAATCTTTGCCACAAATTCTGATTGCGAGCTGATCGTGAA
GGCCATGCAGGGCCTGCTGAAGGACGGCAACCCTATCCCAAGCGCCATC
GCCGCCAATAGCGGAATCTACTGA-3′

The corresponding amino acid sequence for SEQ ID NO: 76 appears below as SEQ ID NO: 77, and in that sequence the following fonts are used:
normal: human Apobec3A
italic: SV40 NLS
underlined: L25-linker
Double underlined: MCP
bold and underlined: mutations W103A, Y131D

SEQ ID NO: 77 is:
MAPKKKRKVEASPASGPRHLMDPHIFTSNFNNGIGRHKTYLCYEVERLD
NGTSVKMDQHRGFLHNQAKNLLCGFYGRHAELRFLDLVPSLQLDPAQIY
RVTWFISWSPCFSAGCAGEVRAFLQENTHVRLRIFAARIYDDDPLYKEA
LQMLRDAGAQVSIMTYDEFKHCWDTFVDHQGCPFQPWDGLDEHSQALSG
RLRAILQNQGNELKTPLGDTTHTSPPCPAPELLGGPMASNFTQFVLVDN
GGTGDVTVAPSNFANGIAEWISSNSRSQAYKVTCSVRQSSAQNRKYTIK
VEVPKGAWRSYLNMELTIPIFATNSDCELIVKAMQGLLKDGNPIPSAIA
ANSGIY

A fourth plasmid, which appears below as SEQ ID NO: 78, encodes for the deaminase fused to MCP, Anolis carolinensis Apobec1a-MCP. Below the following fonts are used:
normal: Anolis Apobec1a
italic: SV40 NLS
underlined: L25-liner
Double underlined: MCP

SEQ ID NO: 78 is
5′-ATGGCCCCCAAGAAGAAGCGGAAAGTGGGGTATCAGGCTGCAATTC
TATTATCAAATTTGTTCTTCAGGTGGCAAATGGAACCAGAGGCGTTTCA
GAGGAATTTTGATCCCAGAGAATTTCCCGAGTGTACTTTACTGCTGTAT
GAAATCCACTGGGATAACAACACCAGTAGGAACTGGTGTACAAACAAAC
CTGGCCTCCATGCTGAAGAAAATTTTTTGCAAATTTTTAATGAGAAAAT
AGATATCAGGCAGGACACACCATGCTCTATCACTTGGTTTCTGTCTTGG
AGTCCTTGTTATCCATGCAGCCAGGCTATAATTAAGTTCTTGGAAGCAC
ACCCTAACGTGAGCCTGGAGATAAAAGCTGCTCGGCTGTACATGCATCA
AATCGACTGTAACAAGGAAGGCCTCAGGAATTTAGGTAGAAATAGAGTT
TCTATCATGAATCTACCTGATTATCGCCACTGTTGGACAACATTTGTGG
TTCCTAGAGGGGCAAATGAAGATTATTGGCCACAGGATTTCTTACCAGC
CATAACAAATTATTCCAGGGAACTTGACTCAATTCTTCAGGACGAGCTG
AAGACACCCCTGGGCGACACCACACACACCTCTCCACCTTGCCCAGCAC
CAGAGCTGCTGGGAGGCCCTATGGCCAGCAACTTCACACAGTTTGTGCT
GGTGGATAATGGAGGAACCGGCGACGTGACAGTGGCACCATCTAACTTT
GCCAATGGCATCGCCGAGTGGATCAGCTCCAACTCTCGGAGCCAGGCCT
ATAAGGTGACCTGTAGCGTGCGGCAGTCTAGCGCCCAGAATAGAAAGTA
TACAATCAAGGTGGAGGTGCCTAAGGGCGCCTGGAGATCCTACCTGAAC
ATGGAGCTGACCATCCCAATCTTTGCCACAAATTCTGATTGCGAGCTGA
TCGTGAAGGCCATGCAGGGCCTGCTGAAGGACGGCAACCCTATCCCAAG
CGCCATCGCCGCCAATAGCGGAATCTACTGA-3′

The corresponding amino acid sequence for SEQ ID NO: 78 appears below as SEQ ID NO: 79, and in that sequence the following fonts are used:
normal: Anolis Apobec1a
italic: SV40 NLS
underlined: L25-linker
Double underlined: MCP

SEQ ID NO: 79 is:
MAPKKKRKVGYQAAILLSNLFFRWQMEPEAFQRNFDPREFPECTLLLYE
IHWDNNTSRNWCTNKPGLHAEENFLQIFNEKIDIRQDTPCSITWFLSWS
PCYPCSQAIIKFLEAHPNVSLEIKAARLYMHQIDCNKEGLRNLGRNRVS
IMNLPDYRHCWTTFVVPRGANEDYWPQDFLPAITNYSRELDSILQDELK
TPLGDTTHTSPPCPAPELLGGPMASNFTQFVLVDNGGTGDVTVAPSNFA
NGIAEWISSNSRSQAYKVTCSVRQSSAQNRKYTIKVEVPKGAWRSYLNM
ELTIPIFATNSDCELIVKAMQGLLKDGNPIPSAIAANSGIY

The gRNA component of the base editing system was expressed on a separate vector with expression being driven by the RNA polymerase III U6 promoter (gRNA expression vector). The gRNA was expressed as a single unit encompassing the crRNA and tracrRNA components of A. acidoterrestris Cas12b or Acidibacillus sulfuroxidans Cas12f linked by an artificial tetra-loop as previously described. A list of gRNA target sequences for each Cas protein are shown in table 5.

TABLE 5
gRNA target site sequences for base editing.

| | | |
|---|---|---|
| Target | Target | |
| name | Target sequence [5′ −> 3′] | SEQ ID NO: |

Cas12b

| | | |
|---|---|---|
| Site2- | TGTTCCAGTTTCCTTTACAG | 80 |
| sgRNA1 | | |
| Site2- | AGGCTGGCCCGCCCCGCAGT | 93 |
| sgRNA2 | | |
| Site2- | CAGCCCGCTGGCCCTGTAAA | 81 |
| sgRNA4 | | |
| VEGFA- | GCCAGAGCCGGGGTGTGCAG | 82 |
| sgRNA1 | | |
| VEGFA- | GGAAGTGTCCAGGGATGCTT | 83 |
| sgRNA6 | | |
| FANCF- | TCGCGCCCTCCCAGCCGGGC | 84 |
| sgRNA3 | | |
| B2M- | CCGATATTCCTCAGGTACTC | 94 |
| sgRNA2 | | |
| B2M- | AGGTTTACTCACGTCATCCA | 95 |
| sgRNA3 | | |

CasMINI

| | | |
|---|---|---|
| VEGFA- | CTCCTGGACCCCCTATTTCTGAC | 85 |
| sgRNA1 | | |
| VEGFA- | GCCAGAGCCGGGGTGTGCAGACG | 86 |
| sgRNA2 | | |

### Cell Culture and Transfection

HEK293 cells were grown in Dulbecco's modified Eagle medium (DMEM) supplemented with 10% fetal bovine serum (FBS) and 100 U m1-1 penicillin/streptomycin. 24 hours prior to transfection 10,000 cells were seeded into a single well of a 96-well plate to achieve ˜70% confluency for transfection. After 24 hours the cells were lipid transfected with 200 ng of plasmid DNA (75 ng Cas vector, 75 ng Deaminase vector, and 50 ng gRNA expression vector) with 0.7 μl of DharmaFECT DUO (Horizon discovery) per well of a 96 well plate.

### Cell Lysis

72 hours after transfection, the medium was removed, and the cells were washed 1× with PBS and 50 μl of TrypLE express enzyme (ThermoFisher scientific) was added to each well. After cells were dissociated, 100 ul of fresh DMEM was added and 20 μl of the resuspended cells were transferred to a 96 well plate and were incubated with 60 μl of DirectPCR lysis reagent (Viagen biotech) under the following conditions: 55° C. for 45 minutes followed by 95° C. for 15 minutes, and then the cell lysates were stored at −20° C.

### PCR Amplification of Targeted Regions

1 μl of cell lysate obtained using the DirectPCR lysis reagent was used per PCR reaction. For NGS analysis, the Q5 high-fidelity 2× master mix (NEB) was used for amplification of sgRNA target sites, reaction mixes were set up as follows:

| Reagent | volume | |
|---|---|---|
| Q5 2x master mix | 12.5 | μl |
| Forward primer (10 μM) | 1.25 | μl |
| Reverse primer (10 μM) | 1.25 | μl |
| Cell lysate | 1.0 | μl |
| Nuclease-free water | 9.0 | μl |
| Total | 25 | μl |

The PCR reaction was performed under the thermocycling conditions identified as follows:

| Step | Temperature | Time | |
|---|---|---|---|
| Initial denaturation | 98° C. | 30 | seconds |
| 30 cycles | 98° C. | 10 | seconds |
| | 64° C./68° C. | 30 | seconds |
| | 72° C. | 30 | seconds |
| Final extension | 72° C. | 2 | minutes |

For Sanger sequencing analysis, regions of interest were PCR-amplified using GoTaq Hot Start polymerase (Promega). Reaction mixes were set up as follows:

| Reagent | Volume | |
|---|---|---|
| 5x GoTaq buffer | 5 | μl |
| MgCl2 (25 mM) | 2 | μl |
| dNTPs (100 mM) | 0.1 | μl |
| Forward primer (10 μM) | 1.25 | μl |
| Reverse primer (10 μM) | 1.25 | μl |
| Nuclease-free water | 15.4 | μl |
| total | 25 | μl |

Primers used are detailed in table 4.

TABLE 6
Primers [5′−> 3′]

| | | |
|---|---|---|
| Site2-F | AGGACGTCTGCCCAATATGT | SEQ ID NO: 87 |
| Site2-R | CCAAGTGAGAAGCCAGTGGA | SEQ ID NO: 88 |
| VEGFA-F | TCTTCCCTCCCAGTCACTGA | SEQ ID NO: 89 |
| VEGFA-R | TCACTCTCGAAGACGCTGCT | SEQ ID NO: 90 |
| FANCF-F | CGATGAGGAGACACTCCAAGAG | SEQ ID NO: 91 |
| FANCF-R | GCCTTTCTGAAGGTCATAGTGC | SEQ ID NO: 92 |

### Base Editing Analysis

PCR products were submitted for Sanger sequencing (Genewiz). Data was analysed by proprietary in-house software (Chimera).

### Example 3: Base Editing using Cas12b

In this example next-generation sequencing analysis of the Site2 amplicon region shows specific C to T transitions introduced by introduction of a Cas12b base editor in an HEK-293T cell line. Two different effectors were used, either hApobec3A (Apobec3A-MCP) or AnoApobec (Anolis carolinensis Apobec1a-MCP), which were introduced through transfection of a plasmid expressing the sequence highlighted in SEQ ID NO: 76 and SEQ ID NO: 78, respectively. Concurrently, dCas12b-UGI-UGI was introduced through transfection of a plasmid expressing the sequence highlighted in SEQ ID NO: 72. gRNAs were introduced through transfection of plasmids expressing gRNA backbones that correspond to SEQ ID NOs: 58 to 65. The target site sequences correspond to SEQ ID NO: 80. The components were delivered by DNA plasmid lipid transfection and the cells were subsequently lysed 72 hours post-transfection, after which the targeted loci were amplified by PCR and analyzed by next-generation sequencing.

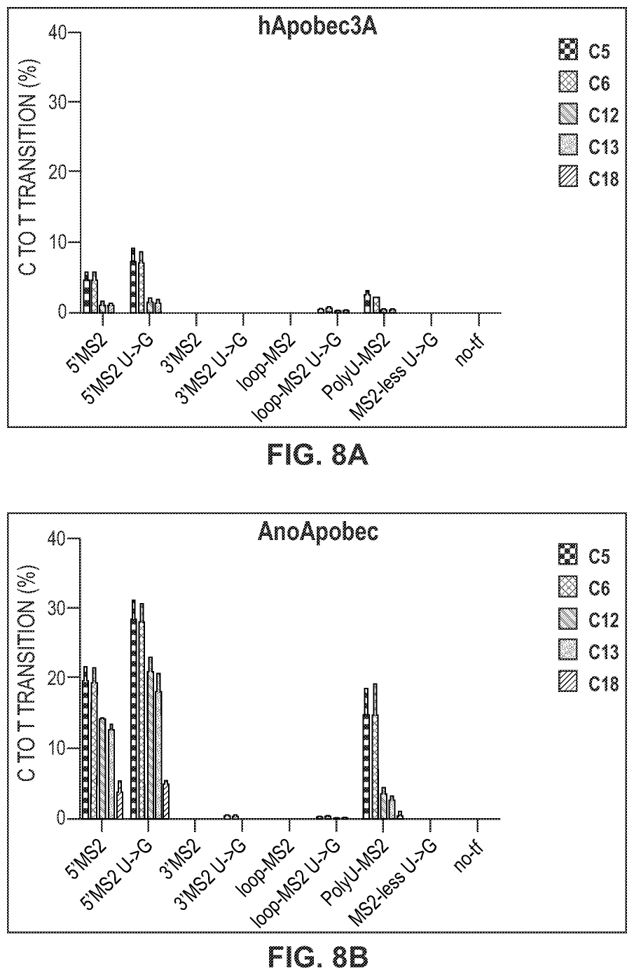

The results are reported in FIG. 8A and FIG. 8B respectively. These figures show the level of C to T transitions achieved by delivery of the base editing components by DNA plasmid transfection in HEK-293 cells. The X-axes denote different locations of the MS2 aptamer in the gRNA and correspond to those sequences highlighted in Table 3. U->G indicates a change in an internal polyU stretch present in the Cas12b gRNA scaffold, introduced to increase efficiency of transcription under a hU6 promoter. The Y axes display the percentage of C to T edits at the specified C residue within the protospacer as measured by NGS. Each bar represents a targeted C residue within the targeted protospacer with the number in the figure legend denoting the position of the relevant C residue with 1 being PAM proximal and 20 PAM distal. No-tf=no transfection control. hApobec3A=Apobec3A-MCP; AnoApobec=Anolis carolinensis Apobec1a-MCP. Error bars represent the standard error of the mean from 2 replicates.

FIG. 8A and FIG. 8B show that the Cas12b base editor is functional in the HEK-293T cell line, with editing observed with both hApobec3A and AnoApobec at Site2. Two gRNA designs are functional with editing observed with both the 5′MS2 design and PolyU-MS2 design, furthermore no editing is apparent in the absence of the MS2 aptamer (MS2-less) indicating the observed editing is dependent on the recruitment of the deaminase through interaction between the MCP ligand and MS2 ligand binding moiety and subsequent assembly of the base editing complex. Editing is observed over a wide window, in a departure from that observed with Cas9 base editors, with up to 5 C residues edited over a 14 nt window. The shifted editing window highlights the general applicability of the tool and its potential to broaden the scope of sites that can be targeted for base editing.

### Example 4: Base Editing in Multiple Sites

This example demonstrates that the Cas12b base editor is functional at multiple genomic loci and sequence contexts. Base editing analysis shows that the Cas12b base editor introduces C to T transitions in different sites in the HEK-293T cell line. Seven different regions were targeted by sgRNAs: VEGFA_sgRNA1, SEQ ID NO: 82; VEGFA_sgRNA6, SEQ ID NO: 83; FANCF_sgRNA3, SEQ ID NO: 84; site2_sgRNA2, SEQ ID NO: 93; site2_sgRNA4, SEQ ID NO: 81; B2M_sgRNA2, SEQ ID NO: 94; and B2M_sgRNA3, SEQ ID NO: 95. Two different effectors were used, either hApobec3A or AnoApobec, which were introduced through transfection of plasmids expressing the sequences that correspond to SEQ ID NO: 76 and SEQ ID NO: 78, respectively. Cas12b was introduced through transfection of a plasmid that expresses the sequence corresponding to SEQ ID NO: 72. A 5′MS2 version of the gRNA (SEQ ID NO: 59) was used for all conditions. The target site sequences correspond to SEQ ID NOs: 93-95, and 81-84. Components were delivered by DNA plasmid lipid transfection and the cells were subsequently lysed 72 hours post-transfection, after which the targeted loci were amplified by PCR and analyzed by Sanger sequencing. Spacer sequences are shown in Table 5 and primers to amplify the edited region are shown in Table 6.

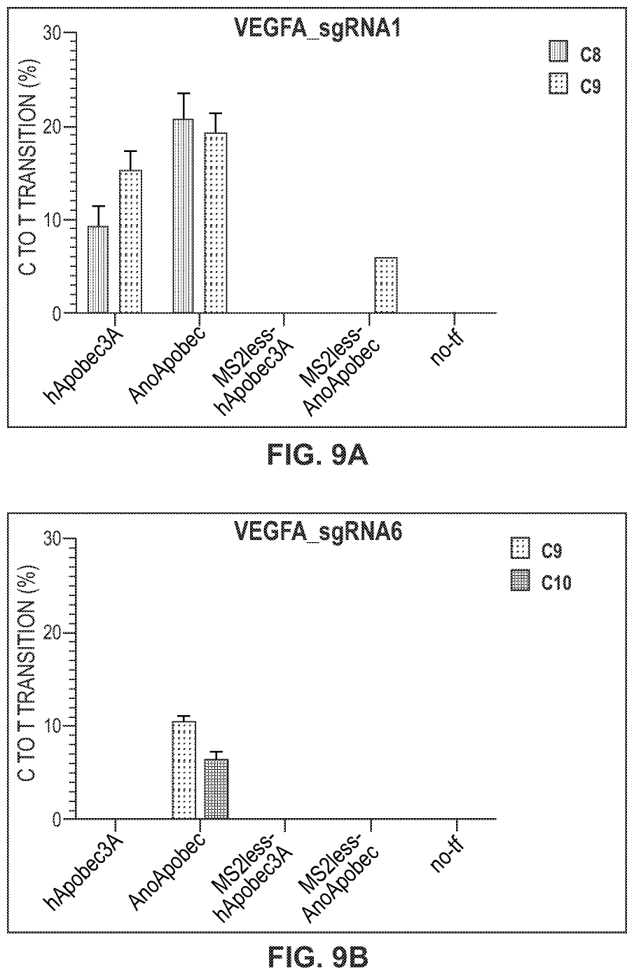

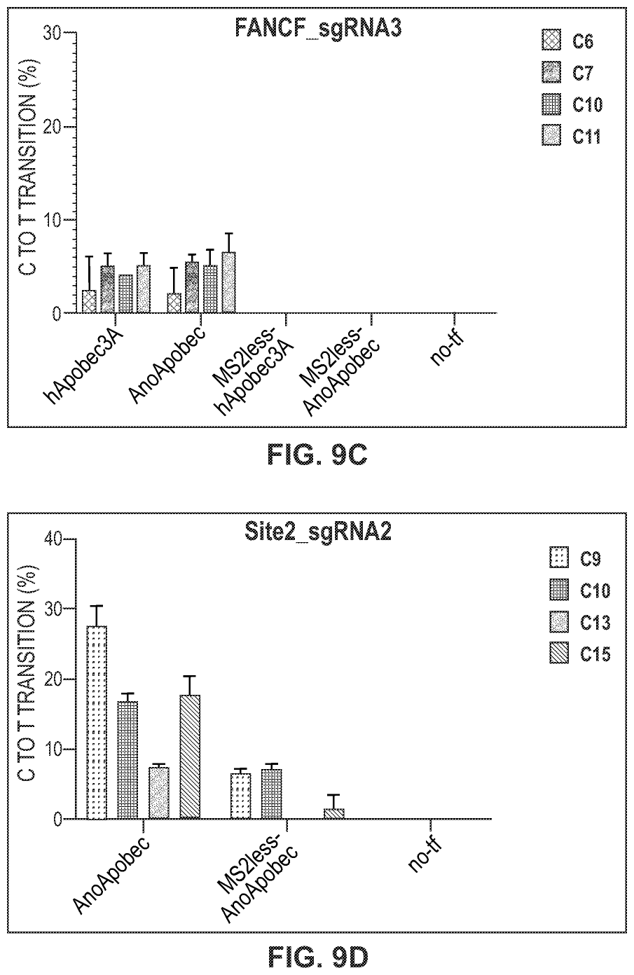

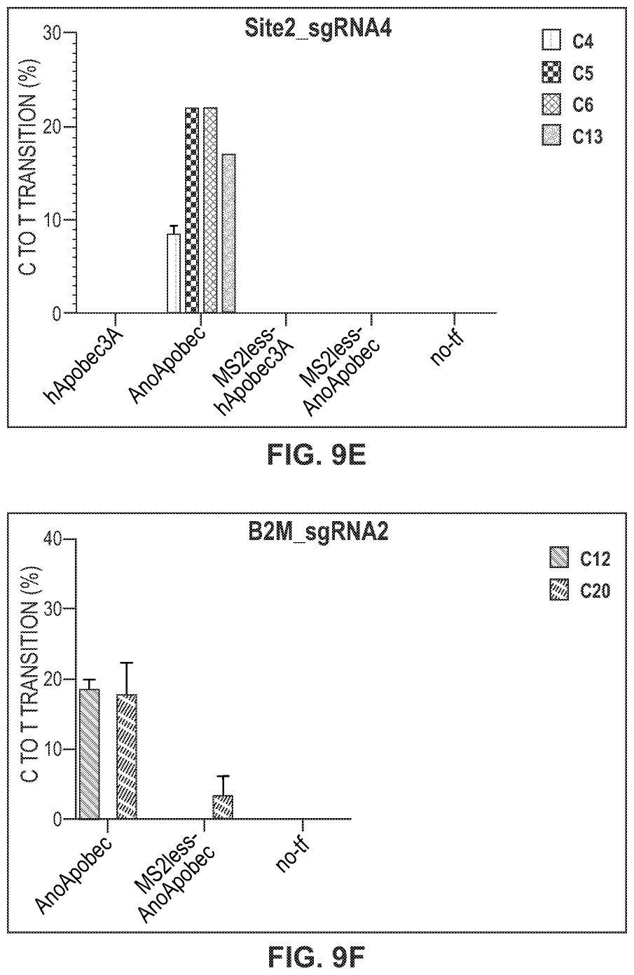

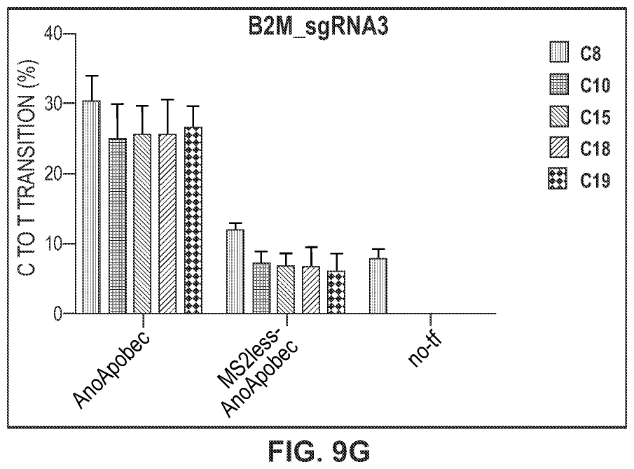

FIG. 9A (VEGFA_sgRNA1), FIG. 9B (VEGFA_sgRNA6), FIG. 9C (FANCF_sgRNA3), FIG. 9D (site2_sgRNA2), FIG. 9E (site2_sgRNA4), FIG. 9F (B2M_sgRNA2), and FIG. 9G (B2M_sgRNA3). Level of C to T transitions achieved by delivery of the base editing components by DNA plasmid transfection in HEK-293 cells at multiple genomic sites and sequence contexts. The X axes denote the reagents used (in conjunction with dCas12b-UGI-UGI); hApobec3A=5′MS2 gRNA+Apobec3A-MCP; AnoApobec=5′MS2 gRNA+Anolis carolinensis Apobec1a-MCP; MS2less-hApobec3A=as hApobec3A except gRNA does not contain MS2 aptamer; AnoApobec=as AnoApobec except gRNA does not contain MS2 aptamer; no-tf=no transfection control. The Y axes display the percentage of C to T edits at the specified C residue within the protospacer as measured by Sanger sequencing and subsequent Chimera analysis. Each bar represents a targeted C residue within the targeted protospacer with the number in the figure legend denoting the position of the relevant C residue with 1 being PAM proximal and 20 PAM distal. Error bars represent the standard error of the mean from 2 replicates.

FIGS. 9A-9G demonstrate that the Cas12b base editor is viable as a general base editing tool, with editing observed at multiple genomic loci and sequence contexts. Again, editing is observed at multiple Cs residues within the protospacer with the editing window size and location differing to that observed with Cas9 base editing systems, and therefore further reinforcing the potential unique functional applications of this tool. Furthermore, in the absence of the MS2 aptamer (MS2less) little-to-no editing was observed indicating that the observed editing is dependent on the recruitment of the deaminase through interaction between the MCP ligand and MS2 ligand binding moiety and subsequent assembly of the base editing complex.

### Example 5: Base Editing in Different Cells Lines

This example demonstrates that the Cas12b base editor is functional in different cell lines with Sanger sequencing and Chimera analysis shows that the Cas12b base editor introduces C to T transitions in the U2OS cell line. One region targeted by one sgRNA is shown, VEGFA_sgRNAL SEQ ID NO: 83. Two different effectors were used, either hApobec3A or AnoApobec, which were introduced through transfection of plasmids that express the sequences that correspond to SEQ ID NO: 76 and SEQ ID NO: 78, respectively. Cas12b was introduced through transfection of a plasmid that expresses the sequence that corresponds to SEQ ID NO: 72. Transfection of plasmid expressing a 5′MS2 version of the sgRNA (SEQ ID NO: 59) was used for all conditions. Components were delivered by DNA plasmid lipid transfection and the cells were subsequently lysed 72 hours post-transfection, after which the targeted loci were amplified by PCR and analyzed by Sanger sequencing.

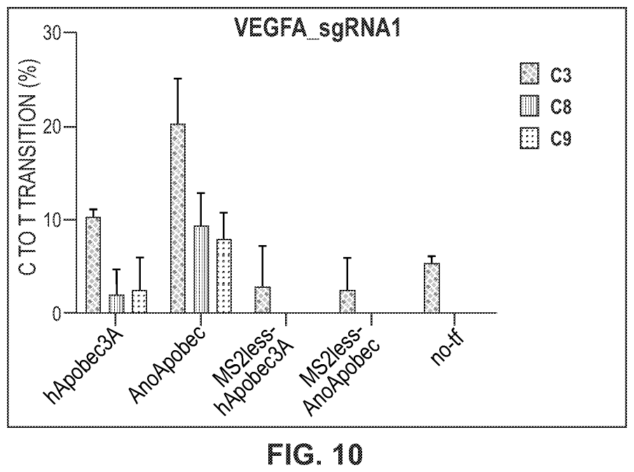

FIG. 10 shows the level of C to T transitions achieved by delivery of the base editing components by DNA plasmid transfection in U2OS cells. Data is shown for the guide VEGFA_sgRNA1, SEQ ID NO: 83. The X axis denotes the reagents used (in conjunction with dCas12b-UGI-UGI); hApobec3A=5′MS2 gRNA+Apobec3A-MCP; AnoApobec=5′MS2 gRNA+Anolis carolinensis Apobec1a-MCP; MS2less-hApobec3A=as hApobec3A except gRNA does not contain MS2 aptamer; AnoApobec=as AnoApobec except gRNA does not contain MS2 aptamer; no-tf=no transfection control. The Y axis displays the percentage of C to T edits at the specified C residue within the protospacer as measured by Sanger sequencing and subsequent Chimera analysis. Each bar represents a targeted C residue within the targeted protospacer with the number in the figure legend denoting the position of the relevant C residue with 1 being PAM proximal and 20 PAM distal. Error bars represent the standard error of the mean from 2 replicates.

FIG. 10 shows that the Cas12b base editor is functional in U2OS cell line for the same sgRNAs as in FIG. 9A, further demonstrating the general applicability of the base editing tool. Editing is observed with both deaminases and, as witnessed in HEK-293 cells, editing is apparent over a broad window.

### Example 6: Base Editing using CasMINI

This example demonstrates that the gRNA-ligand base editing system can be applied to other Type V enzymes other than Cas12b. Here it is shown that a CasMINI base editor introduces C to T transitions in different sites in HEK-293T cells. Two different effectors were used, either hApobec3A or AnoApobec, which were introduced through transfection of plasmids that express sequences corresponding to SEQ ID NO: 76 and SEQ ID NO: 78, respectively. CasMINI was introduced through transfection of a plasmid that expresses the sequence that corresponds to SEQ ID NO: 74. gRNAs were introduced through transfection of plasmids expressing gRNA backbones that correspond to SEQ ID NOs: 66 to 71. Two different regions targeted by two different sgRNAs are shown; VEGFA_sgRNA1, SEQ ID NO: 85, and VEGFA_sgRNA2, SEQ ID NO: 86. Components were delivered by DNA plasmid lipid transfection and the cells were subsequently lysed 72 hours post-transfection, after which the targeted loci were amplified by PCR and analyzed by Sanger sequencing.

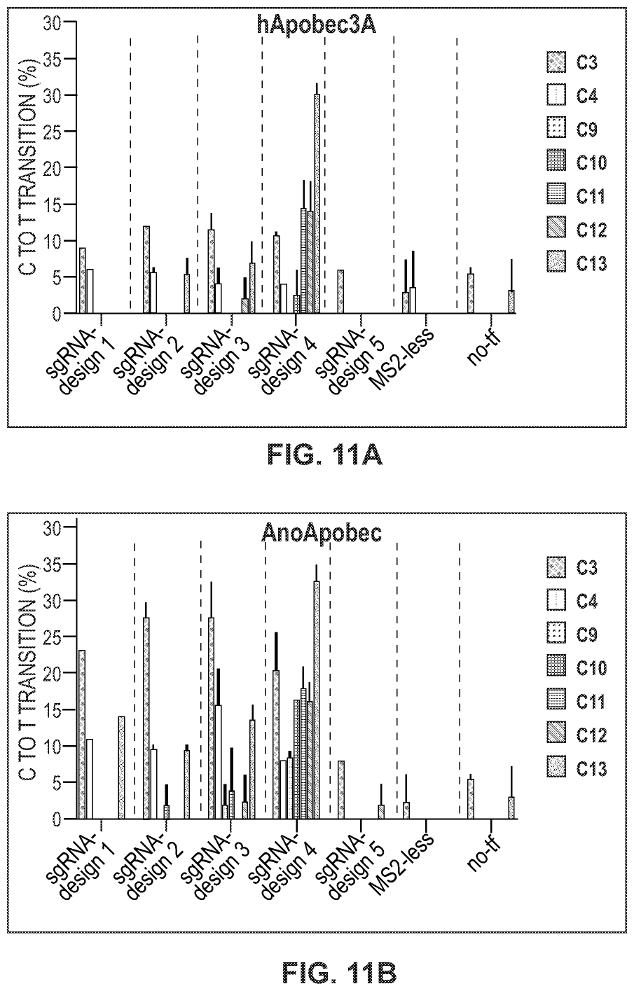

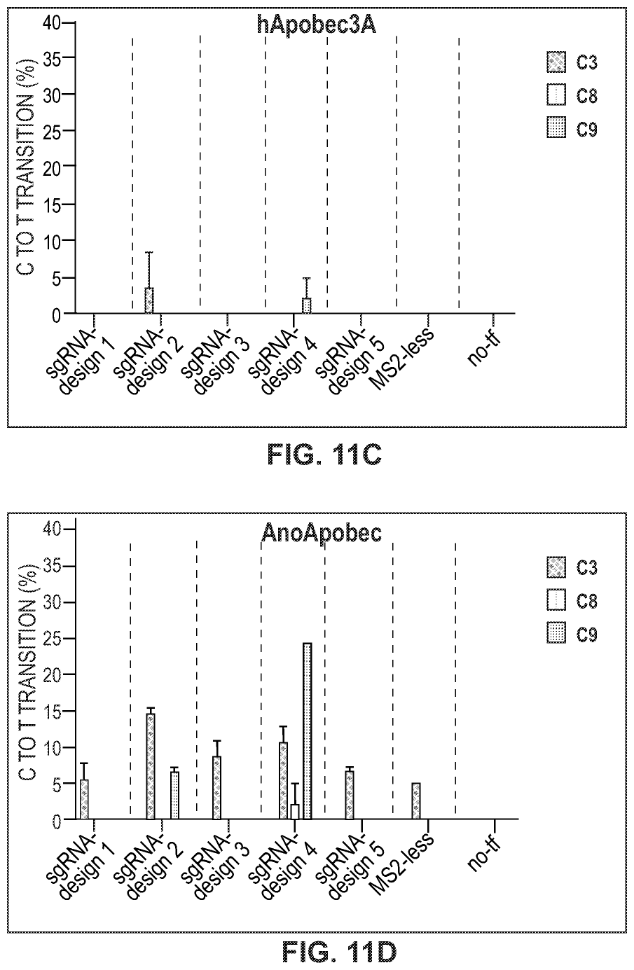

FIGS. 11A-11D. Level of C to T transitions achieved by delivery of the CasMINI base editing components by DNA plasmid transfection in HEK-293 cells. The X-axes denote different locations of the MS2 aptamer in the gRNA and correspond to those sequences highlighted in Table 4; sgRNA-design 1, SEQ ID NO: 67, corresponds to a 5′MS2, sgRNA-design 2, SEQ ID NO: 68, corresponds to 5′MS2 and a stem loop 1 truncation, sgRNA-design 3, SEQ ID NO: 69, corresponds to an MS2 located as an extension of stem loop 1, sgRNA-design 4, SEQ ID NO: 70, corresponds to a MS2 located as a replacement of stem loop 2 and a truncation of stem loop 1, sgRNA-design 5, SEQ ID NO: 71, corresponds to a MS2 located as a replacement of the repeat:antirepeat region. MS-2less in these figures corresponds to SEQ ID NO: 66. The Y axes display the percentage of C to T edits at the specified C residue within the protospacer as measured by Sanger sequencing and subsequent Chimera analysis. Each bar represents a targeted C residue within the targeted protospacer with the number in the figure legend denoting the position of the relevant C residue with 1 being PAM proximal and 20 PAM distal. Error bars represent the standard error of the mean from 2 replicates.

FIGS. 11A, 11B, 11C, and 11D show that CasMINI base editor is functional in HEK-293T cells. As demonstrated for Cas12b, both AnoApobec and hApobec3a are functional as part of the CasMINI base editor and 3 different gRNA designs are consistently functional (designs 2, 3 and 4). Intriguingly the placement of the ligand binding moiety had a clear impact on editing behavior, with design 4 demonstrating a wide editing window and broad elevated levels of editing, whilst designs 2 and 3 show a marked preference for a particular C residue within the protospacer. This ability to alter editing windows (both positionally and their size) through alternative placement of the ligand binding moiety within the gRNA further exemplifies the unique practical application and flexibility of the gRNA-ligand base editing system. Once again little-to-no editing was observed in the absence of the MS2 aptamer (MS2-less) indicating that the observed editing is dependent on the recruitment of the deaminase through interaction between the MCP ligand and MS2 ligand binding moiety and subsequent assembly of the base editing complex.

1. A gRNA-ligand binding complex, wherein the gRNA-ligand binding complex comprises:
a. a gRNA, wherein the gRNA contains 60 to 210 nucleotides and the gRNA comprises
i. a crRNA sequence, wherein the crRNA sequence is 35 to 60 nucleotides long and the crRNA sequence comprises a Cas association region, wherein the Cas association region is 18 to 30 nucleotides long and a targeting region, wherein the targeting region is 18 to 30 nucleotides long, and
ii. a tracrRNA sequence, wherein the tracrRNA sequence is 45 to 120 nucleotides long and wherein the tracrRNA sequence comprises an anti-repeat region and a distal region, wherein the anti-repeat region is at least 80% complementary to the Cas association region over at least 18 consecutive nucleotides of the Cas association region, and the Cas association region and the anti-repeat region are capable of hybridizing to form a hybridization region, wherein the hybridization region is capable of retaining association with an RNA binding domain of a Type V Cas protein; and
b. a ligand binding moiety, wherein the ligand binding moiety is either (i) directly bound to the gRNA, or (ii) bound to the gRNA through a linker.

2. The gRNA-ligand binding complex of claim 1, wherein the gRNA is formed by a single strand of nucleotides and the single strand of nucleotides comprises the crRNA sequence and the tracrRNA sequence and wherein between the Cas association region and the anti-repeat region, there is a loop sequence.

3. (canceled)

4. (canceled)

5. The gRNA-ligand binding complex of claim 1, wherein the gRNA is comprised of a first strand of nucleotides and a second strand of nucleotides, wherein the first strand of nucleotides comprises the tracrRNA sequence and the second strand of nucleotides comprises the crRNA sequence.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The gRNA-ligand binding complex of claim 2, wherein the gRNA has a 5′ end, and the ligand binding moiety is directly bound to the gRNA at the 5′ end of the gRNA.

24. The gRNA-ligand binding complex of claim 2, the gRNA has a 3′ end, and the ligand binding moiety is directly bound to the gRNA at the 3′ end of the gRNA.

25. The gRNA-ligand binding complex of claim 2, wherein the gRNA has a 3′ end and a 5′ end, and the ligand binding moiety is directly bound to the gRNA at a nucleotide other than at the 3′ end or the 5′ end.

26. (canceled)

27. (canceled)

28. (canceled)

29. (canceled)

30. The gRNA-ligand binding complex of claim 2, wherein the ligand binding moiety is directly bound to the gRNA at the loop sequence.

31. The gRNA-ligand binding complex of claim 1, wherein the gRNA comprises a loop sequence and the ligand binding moiety is directly bound to the gRNA at the loop sequence.

32. The gRNA-ligand binding complex of claim 1, wherein the gRNA-ligand binding complex comprises the linker and the ligand binding moiety is bound to the gRNA through the linker.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The gRNA-ligand binding complex of claim 1, wherein the ligand binding moiety is able to associate with a ligand from the group consisting of: MS2, Ku, PP7, SfMu, Sm7, Tat, Glutathione S-transferase (GST), CSY4, Qbeta, COM, pumilio, Anti-His Tag (6H7), lambda N22plus, SNAP-Tag, a lectin, and PDGF beta-chain.

49. (canceled)

50. (canceled)

51. The gRNA-ligand binding complex of claim 32, wherein the ligand binding moiety is a first ligand binding moiety and the gRNA-ligand binding complex comprises a second ligand binding moiety, and wherein the linker is a first linker and the gRNA-ligand binding complex comprises a second linker, wherein the first ligand binding moiety is attached to the first linker and the second ligand binding moiety is attached to the second linker.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. A base editing complex comprising:
a. the gRNA-ligand binding complex of claim 1; and
b. a Type V Cas protein, wherein the hybridization region of the gRNA-ligand binding complex is associated with the Type V Cas protein.

65. (canceled)

66. The base editing complex of claim 64, wherein the Type V Cas protein comprises an active RuvC domain.

67. The base editing complex of claim 64, wherein the Type V Cas protein comprises a deactivated RuvC domain.

68. (canceled)

69. The base editing complex of any claim 64 further comprising an effector, wherein the effector is attached to a ligand binding moiety ligand and the ligand binding moiety ligand is capable of associating with the ligand binding moiety.

70. The base editing complex of claim 69, wherein the effector is selected from the group consisting of deaminases, reverse transcriptases, transcriptional regulators and repair enzymes.

71. (canceled)

72. (canceled)

73. (canceled)

74. (canceled)

75. (canceled)

76. (canceled)

77. A method for base editing comprising exposing the base editing complex of claim 64 to double-stranded DNA.

78. (canceled)

79. (canceled)

80. (canceled)

81. (canceled)

82. (canceled)

83. (canceled)

84. (canceled)

85. A method editing DNA in a cell, said method comprising exposing the base editing complex of claim 64 to the cell.

86. A population of genetically edited cells obtained according to the method of claim 85.

87. (canceled)

88. (canceled)

89. A method of treating a subject, said method comprising the method of claim 77, wherein said exposing takes place outside of a subject and after said exposing, infusing the cell into the subject.
